(19) United States
(12) Patent Application Publication
(10) Pub. No.: US 2016/0289655 A1
(43) Pub. Date: Oct. 6, 2016

(54) Thermostable Phytase Variants

(71) Applicant: NOVOZYMES A/S, Bagsvaerd (DK)

(72) Inventors: Leonardo De Maria, Frederiksberg (DK); Lars Kobberoee Skov, Bagsvaerd (DK); Michael Skjoet, Roskilde (DK)

(73) Assignee: Novozymes A/S, Bagsvaerd (DK)

(21) Appl. No.: 15/185,250

(22) Filed: Jun. 17, 2016

Related U.S. Application Data
(63) Continuation of application No. 13/636,199, filed on Sep. 20, 2012.
(60) Provisional application No. 61/318,011, filed on Mar. 26, 2010.

(30) Foreign Application Priority Data
Mar. 26, 2010 (EP) 10158031.4

(51) Int. Cl. C12N 9/16; A23K 20/189; A23K 50/80; A23K 50/10; A23K 50/30; A23K 50/75; C12P 7/06; A23K 20/147

(52) U.S. Cl. CPC C12N 9/16; C12P 7/06; A23K 20/189; A23K 20/147; A23K 50/10; A23K 50/30; A23K 50/75; A23K 50/80; A23L 1/302; A23L 1/304; A23L 1/30; A23V 2002/00

(57) ABSTRACT

The present invention relates to a phytase which has at least 70% identity to a phytase derived from E. coli and comprises at least one modification as compared to this phytase. These phytase variants have modified, preferably improved, properties, such as thermostability, temperature profile, pH profile, specific activity, performance in animal feed, reduced protease sensitiliby, and/or an modified glycosylation pattern. The invention also relates to DNA encoding these phytases, methods of their production, as well as the use thereof, e.g., in animal feed and animal feed additives.

### CROSS-REFERENCE TO RELATED APPLICATIONS

This application is a continuation of U.S. application Ser. No. 13/636,199 filed Sep. 20, 2012, which is a 35 U.S.C. 371 national application of international application no. PCT/EP2011/054652 filed Mar. 25, 2011, which claims priority or the benefit under 35 U.S.C. 119 of European application no. 10158031.4 filed Mar. 26, 2010 and U.S. provisional application No. 61/318,011 filed Mar. 26, 2010. The content of these applications is fully incorporated herein by reference.

2. Field of the Invention

The present invention relates to a method of producing a variant phytase having at least 70% identity to SEQ ID NO:2 and comprising the establishment of at least one disulfide bridge as compared to SEQ ID NO:2, wherein said at least one disulfide bridge is not among the four naturally occurring ones in positions 77/108, 133/408, 178/188, and 382/391 with the numbering as provided in SEQ ID NO:2 and closely related phytases (i.e., is a variant thereof). The invention also relates to DNA encoding these phytases, methods of their production, as well as the use thereof, e.g., in animal feed and animal feed additives. The mature part of the Escherichia coli phytase reference is included in the sequence listing as SEQ ID NO:2. The reference phytase is the one deposited in UNIPROT under the id. A7ZK83.

### BACKGROUND OF THE INVENTION

1. Background Art

Phytases are well-known enzymes, as are the advantages of adding them to foodstuffs for animals, including humans. Phytases have been isolated from various sources, including a number of fungal and bacterial strains.

It is an object of the present invention to provide alternative polypeptides having phytase activity (phytases) and polynucleotides encoding the polypeptides. The phytase variants of the invention exhibit modified or altered preferably improved properties as compared to the parent phytase. Non-limiting examples of such properties are: Stability (such as acid-stability, heat-stability, steam stability, pelleting stability, and/or protease stability, in particular pepsin stability), temperature profile, pH profile, specific activity, substrate specificity, performance in animal feed(such as an improved release and/or degradation of phytate), susceptibility to glycation, and/or glycosylation pattern.

As described herein, mutagenesis of a parent polynucleotide encoding a phytase is employed to prepare variant (synthetic) DNAs encoding a phytase having improved properties relative to the phytase encoded by the parent polynucleotide.

A number of three-dimensional structures of phytases of the Histidine acid phosphate (HAP) type are known. (e.g., Lim et al., 2000, Nature Struct. Biol. 7: 108-113). From these it has been found that they all have four disulfide bridges located at position pairs 77/108 133/407 178/187 381/390 (according to the numbering used here). Typically these occupy all the cysteines present in the molecule.

### Escherichia

The sequence of the appA gene from E. coli was published by Dassa et al. (J. Bacteriol. 172(9): 5497-5500 (1990)). The E. coli phytase comprises the four disulfide bridges indicated above and no further cysteines are present.

Variants of the E. coli phytase were first disclosed in Ostanin et al. (J. Biol. Chem. 267(32): 22830-22836 (1992)) [R16A, H17N, R20A, R92A, H303A, R63A] showing that the replacement of the R16 and H17 was detrimental to the activity and also that the replacement of the R20, R92 and H303 reduced the activity very strongly, whereas the R63 replacement provided a minor positive effect on activity.

Other variants of the E. coli phytase have been disclosed in WO 2006/028684 (Diversa Corp.), WO 2009/073399 (Syngenta Part. AG), WO 2008/036916 (Verenium Inc.), WO 02/095003 (Diversa Corp.).

It is an object of the invention to provide phytases of modified, preferably, improved temperature properties. Non-limiting examples of such properties are: Thermostability, and temperature profile.

### BRIEF DESCRIPTION OF THE DRAWINGS AND SEQUENCE LISTING

FIG. 1 is an alignment of the phytases of SEQ ID NOs:2 to 6. The position numbers in FIG. 1 refer to the numbering of SEQ ID NO:2.

In the FIGURE (Sequence listing) the sequences apply as follows:

| | |
|---|---|
| SEQ ID NO: 1 | DNA encoding SEQ ID NO: 2 |
| SEQ ID NO: 2 | E. coli phytase wild-type |
| SEQ ID NO: 3 | Wild-type from WO 2009/073399 |
| SEQ ID NO: 4 | Variant Nov9X from WO 2009/073399 |
| SEQ ID NO: 5 | SEQ ID NO: 2 from WO 2008/036916 |
| | (18 and 323 are Xaa) |
| SEQ ID NO: 6 | SEQ ID NO: 4 from WO 2008/036916 |

### SUMMARY OF EXAMPLES

In the specification the following examples are provided:

Example 1: Preparation of variants, and determination of activity

Example 2: Specific activity

Example 3. Thermostability

Example 4. Temperature profile

Example 5. pH profile

Example 6: Performance in animal feed in an in vitro model

Example 7: Performance in an in vivo pig trial

### DETAILED DESCRIPTION OF THE INVENTION

In a first aspect, the present invention relates to a method of producing a variant phytase having at least 70% identity to SEQ ID NO:2 and comprising the establishment of at least one disulfide bridge as compared to SEQ ID NO:2, wherein said at least one disulfide bridge is not among the four naturally occurring ones in positions 77/108, 133/408, 178/188, and 382/391 with the numbering as provided in SEQ ID NO:2.

The percentage of identity is determined as described in the section “Phytase Polypeptides, Percentage of Identity”.

The position numbers refer to the position numbering of SEQ ID NO:2, as described in the section “Position Numbering.” Positions corresponding to these SEQ ID NO:2 position numbers in other phytases are determined as described in the section “Identifying Corresponding Position Numbers.”

The at least one disulfide bridge is established in one or more positions selected from the group consisting of the position pairs: A) 52C/99C, B) 141C/200C, C) 31C/177C, D) 91C/46C, E) 31C/176C, F) 59C/100C, and G) 162C/248C.

According to the invention a first disulfide bridge is preferably established in the position pair A between the residues in positions 52 and 99 and a second disulfide bridge is established in the position pair B between the residues in positions 141 and 200.

In certain embodiments the number of established disulfide bridges is 2, 3, 4, 5, 6, and/or 7.

When the number of established disulfide bridges is two the following combinations of position pairs may be created: A+B, A+C, A+D, A+E, A+F, A+G, B+C, B+D, B+E, B+F, B+G, C+D, C+E, C+F, C+G, D+E, D+F, D+G, E+F, E+G, and F+G.

When the number of established disulfide bridges is three the following combinations of position pairs may be created: A+B+C, A+B+D, A+B+E, A+B+F, A+B+G, A+C+D, A+C+E, A+C+F, A+C+G, A+D+E, A+D+F, A+D+G, A+E+F, A+E+G, A+F+G, B+C+D, B+C+E, B+C+F, B+C+G, B+D+E, B+D+F, B+D+G, B+E+F, B+E+G, B+F+G, C+D+E, C+D+F, C+D+G, C+E+F, C+E+G, C+F+G, D+E+F, D+E+G, D+F+G, and E+F+G.

When the number of established disulfide bridges is four the following combinations of position pairs may be created: A+B+C+D, A+B+C+E, A+B+C+F, A+B+C+G, A+B+D+E, A+B+D+F, A+B+D+G, A+B+E+F, A+B+E+G, A+B+F+G, A+C+D+E, A+C+D+F, A+C+D+G, A+C+E+F, A+C+E+G, A+C+E+H, A+C+F+G, A+D+E+F, A+D+E+G, A+D+F+G, A+E+F+G, B+C+D+E, B+C+D+F, B+C+D+G, B+C+E+F, B+C+E+G, B+C+F+G, B+D+E+F, B+D+E+G, B+D+F+G, B+E+F+G, C+D+E+F, C+D+E+G, C+D+F+G, C+E+F+G, C+E+F+H, and D+E+F+G.

When the number of established disulfide bridges is five the following combinations of position pairs may be created: A+B+C+D+E, A+B+C+D+F, A+B+C+D+G, A+B+C+E+F, A+B+C+E+G, A+B+C+F+G, A+B+D+E+F, A+B+D+E+G, A+B+D+F+G, A+B+E+F+G, A+B+F+G+H, A+C+D+E+F, A+C+D+E+G, A+C+D+F+G, A+C+E+F+G, A+D+E+F+G, B+C+D+E+F, B+C+D+E+G, B+C+D+F+G, B+C+E+F+G, B+D+E+F+G, and C+D+E+F+G.

When the number of established disulfide bridges is six the following combinations of position pairs may be created: A+B+C+D+E+F, A+B+C+D+E+G, A+B+C+D+F+G, A+B+C+E+F+G, A+B+D+E+F+G, A+C+D+E+F+G, and B+C+D+E+F+G.

When the number of established disulfide bridges is seven the following combination of position pairs may be created: A+B+C+D+E+F+G.

In all of the above combinations A means 52C/99C, B means 141C/200C, C means 31C/177C, D means 91C/46C, E means 31C/176C, F means 59C/100C, and G means 162C/248C.

According to the method of the invention the phytase variant may further comprise at least one modification in at least one position selected from the following: 25, 37, 38, 75, 77, 108, 114, 123, 126, 127, 133, 137, 141, 142, 146, 157, 173, 178, 188, 204, 211, 233, 235, 253, 267, 286, 287, 317, 318, 327, 367, 382, 391, and 408.

The invention further provides that the above modifications specifically are chosen from the following modifications: 25F, 37F, 38Y, 75K, 75V, 75E, 77A, 108A, 114H, 123E, 126Y, 127L, 127V, 133A, 137E, 137V, 141R, 142R, 146R, 157R, 173Y, 178A, 188A, 204N, 204D, 211W, 233E, 235Y, 253V, 267A, 286F, 287Y, 317L, 318Y, 327Y, 367F, 382A, 391A, and 408A.

In specific embodiments of the invention the additional disulfide bridges are selected from the group comprising: G52C/A99C, T141C/V200C, D31C/L177C, E91C/W46C, D31C/N176C, G59C/F100C, and A162C/S248C.

In further specific embodiments of the method of the invention further specific modifications are selected from:

A25F, W37F, P38Y, C75K, C75V, C75E, C77A, C108A, T114H, P123E, N126Y, P127L, P127V, C133A, N137E, N137V, T141R, D142R, E146R, G157R, P173Y, C178A, C188A, C204N, C204D, V211W, G233E, G235Y, Q253V, R267A, K286F, Q287Y, N317L, W318Y, T327Y, S367F, C382A, C391A, C408A, and/or from the following combinations W37F/P38Y, P123E/P127L, N126Y/P127V, G233E/G235Y, K286F/Q287Y, N317L/W318Y, W37F/P38Y/P123E/P127L, W37F/P38Y/N126Y/P127V, P173Y/N317L/W318Y, C75K/C204N, C75K/C204N/C178A/C188A, C75K/C204N/C382A/C391A, C75K/C204N/C178A/C188A/C382A/C391A, C75K/C204N/C77A/C108A, C75K/C204N/C133A/C108A A25F/C75V/T114H/N137E/T141R/E146R/G157R/C204D/V211W/Q253V/R267A/T327Y/C178A/C188A, A25F/C75V/T114H/N137E/T141R/E146R/G157R/C204D/V211W/Q253V/R267A/T327Y/C382A/C391A, A25F/C75V/T114H/N137E/T141R/E146R/G157R/C204D/V211W/Q253V/R267A/T327Y/C178A/C188A/C382A/C391A, A25F/C75V/T114H/N137E/T141R/E146R/G157R/C204D/V211W/Q253V/R267A/T327Y/C77A/C108A, A25F/C75V/T114H/N137E/T141R/E146R/G157R/C204D/V211W/Q253V/R267A/T327Y/C133A/C408A, A25F/C75E/T114H/N137V/G157R/C204D/V211W/Q253V/R267A/T327Y, A25F/C75E/N137V/T141R/D142R/G157R/C204 D/V211W/Q253V/T327Y, A25F/T114H/N137V/T141R/D142R/G157R/C204D/V211W/Q253V/T327Y, A25F/T114H/N137V/T141R/D142R/E146R/G157R/C204D/V211W/Q253V/R267A/T327Y, A25F/C75V/T114H/N137V/T141R/E146R/G157R/C204D/V211W/Q253V/R267A/T327Y, A25F/C75V/T114H/N137V/T141R/D142R/E146R/G157R/C204D/V211W/Q253V/T327Y, A25F/T114H/N137V/T141R/D142R/E146R/G157R/C204D/V211W/Q253V/T327Y, A25F/C75E/T114H/N137V/T141R/D142R/G157R/C204D/V211W/Q253V/T327Y, A25F/C75E/T114H/N137V/T141R/D142R/E146R/G157R/V211W/Q253V/R267A/T327Y, A25F/C75E/T114H/N137E/T141R/D142R/G157R/V211W/Q253V/R267A/T327Y/L341P, A47F/C97V/T136H/N159V/T163R/D164R/G179R/V233W/Q275V/R289A/T349Y, A25F/C75E/T114H/N137V/T141R/D142R/E146R/G157R/C204D/V211W/Q253V/R267A/T327Y, A25F/C75E/N137V/D142R/G157R/C204 D/V211W/Q253V/R267A/T327Y, A25F/C75E/N137E/T141R/D142R/E146R/G157R/C204D/V211W/Q253V/R267A/T327Y, A25F/C75V/T114H/N137E/T141R/D142R/E146R/G157R/C204D/V211W/Q253V/R267A/T327Y, A25F/C75E/T114H/N137V/T141R/G157R/C204D/V211W/Q253V/T327Y, A25F/C75E/T114H/N137V/T141R/D142R/E146R/G157R/C204D/V211W/Q253V/T327Y, A25F/T114H/N137E/T141R/D142R/G157R/C204D/V211W/Q253V/R267A/T327Y, A25F/T114H/N137V/D142R/G157R/C204D/V211W/Q253V/T327Y, A25F/C75V/T114H/N137V/D142R/G157R/C204 D/V211W/Q253V/T327Y, A25F/C75V/T114H/N137V/D142R/G157R/V211W/Q253V/T327Y, A25F/T114H/N137V/C204D/V211W/T327Y.

The method of the invention may be used to create a variant of any wild-type or variant phytase. In particular embodiments, it produces a variant of the mature part of the phytase of of SEQ ID NO:2, SEQ ID NO:3, SEQ ID NO:4, SEQ ID NO:5, or SEQ ID NO:6 is used as a parent/backbone for producing a phytase variant.

The method of the invention may provide a phytase variant having improved properties, such as thermostability, heat-stability, steam stability, temperature profile, pelleting stability, acid-stability, pH profile, and/or protease stability, in particular pepsin stability, specific activity, substrate specificity, performance in animal feed (such as an improved release and/or degradation of phytate), susceptibility to glycation, and/or glycosylation pattern. The variants provided by the invention exhibit especially improved thermal properties, such as thermostability, heat-stability, steam stability, temperature profile, pelleting stability or improved performance in animal feed.

The method of the invention thus relates to phytase variants having improved thermal properties, such as thermostability, heat-stability, steam stability, temperature profile, and/or pelleting stability.

The method of the invention thus relates to phytase variants having improved thermostability.

The method of the invention thus relates to phytase variants having improved heat-stability.

The method of the invention thus relates to phytase variants having improved steam stability.

The method of the invention thus relates to phytase variants having improved temperature profile.

The method of the invention thus relates to phytase variants having improved pelleting stability.

The method of the invention thus relates to phytase variants having improved acid-stability.

The method of the invention thus relates to phytase variants having improved pH profile.

The method of the invention thus relates to phytase variants having improved protease stability, in particular pepsin stability.

The method of the invention thus relates to phytase variants having improved specific activity.

The method of the invention thus relates to phytase variants having improved substrate specificity.

The method of the invention thus relates to phytase variants having improved performance in animal feed (such as an improved release and/or degradation of phytate).

The method of the invention thus relates to phytase variants having improved susceptibility to glycation.

The method of the invention thus relates to phytase variants having improved and/or glycosylation pattern.

The invention further relates to polynucleotide comprising nucleotide sequences which encode the phytase variants produced by the method, nucleic acid constructs comprising the polynucleotides operably linked to one or more control sequences that direct the production of the polypeptide in an expression host, recombinant expression vectors comprising such nucleic acid constructs, and recombinant host cells comprising a nucleic acid construct and/or an expression vector.

The invention further relates to methods for producing phytase variants as provided comprising

(a) cultivating a host cell to produce a supernatant comprising the phytase; and

(b) recovering the phytase.

The invention further relates to transgenic plants, or plant part, capable of expressing the phytase variants, compositions comprising at least one phytase variant, and (a) at least one fat soluble vitamin; (b) at least one water soluble vitamin; and/or (c) at least one trace mineral. Such compositions may further comprise at least one enzyme selected from the following group of enzymes: amylase, phytase, phosphatase, xylanase, galactanase, alpha-galactosidase, protease, phospholipase, and/or beta-glucanase. The compositions may be animal feed additives that may have a crude protein content of 50 to 800 g/kg and comprising a phytase variant of the invention.

The invention further relates to methods for improving the nutritional value of an animal feed, by adding a phytase variant of the invention to the feed, processes for reducing phytate levels in animal manure by feeding an animal with an effective amount of the feed, methods for the treatment of vegetable proteins, comprising the step of adding a phytase variant to at least one vegetable protein, and the use of a phytase variant of a composition of the invention.

The invention also provides a method for producing a fermentation product such as, e.g., ethanol, beer, wine, comprising fermenting a carbohydrate material in the presence of a phytase variant, a method for producing ethanol comprising fermenting a carbohydrate material in the presence of a phytase variant and producing ethanol.

### Phytase Polypeptides, Percentage of Identity

In the present context a phytase is a polypeptide having phytase activity, i.e., an enzyme which catalyzes the hydrolysis of phytate (myo-inositol hexakisphosphate) to (1) myo-inositol and/or (2) mono-, di-, tri-, tetra- and/or penta-phosphates thereof and (3) inorganic phosphate.

In the present context the term a phytase substrate encompasses, i.a., phytic acid and any phytate (salt of phytic acid), as well as the phosphates listed under (2) above.

The ENZYME site at the internet (expasy.ch/enzyme) is a repository of information relative to the nomenclature of enzymes. It is primarily based on the recommendations of the Nomenclature Committee of the International Union of Biochemistry and Molecular Biology (IUB-MB) and it describes each type of characterized enzyme for which an EC (Enzyme Commission) number has been provided (Bairoch, 2000, The ENZYME database, Nucleic Acids Res. 28:304-305). See also the handbook Enzyme Nomenclature from NC-IUBMB, 1992).

According to the ENZYME site, three different types of phytases are known: A so-called 3-phytase (alternative name 1-phytase; a myo-inositol hexaphosphate 3-phosphohydrolase, EC 3.1.3.8), a so-called 4-phytase (alternative name 6-phytase, name based on 1L-numbering system and not 1D-numbering, EC 3.1.3.26), and a so-called 5-phytase (EC 3.1.3.72). For the purposes of the present invention, all three types are included in the definition of phytase.

In a particular embodiment, the phytases of the invention belong to the family of acid histidine phosphatases, which includes the Escherichia coli pH 2.5 acid phosphatase (gene appA) as well as fungal phytases such as Aspergillus awamorii phytases A and B (EC: 3.1.3.8) (gene phyA and phyB). The histidine acid phosphatases share two regions of sequence similarity, each centered around a conserved histidine residue. These two histidines seem to be involved in the enzymes' catalytic mechanism. The first histidine is located in the N-terminal section and forms a phosphor-histidine intermediate while the second is located in the C-terminal section and possibly acts as proton donor.

In a further particular embodiment, the phytases of the invention have a conserved active site motif, viz. R-H-G-X-R-X-P, wherein X designates any amino acid (see amino acids 16 to 22 of SEQ ID NOs: 2, 3, 4, 6 and amino acids 38-44 of SEQ ID NO: 9). In a preferred embodiment, the conserved active site motif is R-H-G-V-R-A-P, i.e., amino acids 16-22 (by reference to SEQ ID NO: 2) are RHGVRAP.

For the purposes of the present invention the phytase activity is determined in the unit of FYT, one FYT being the amount of enzyme that liberates 1 micro-mol inorganic ortho-phosphate per min. under the following conditions: pH 5.5; temperature 37° C.; substrate: sodium phytate (C₆H₆O₂₄P₆Na₁₂) in a concentration of 0.0050 mol/l. Suitable phytase assays are the FYT and FTU assays described in Example 1 of WO 00/20569. FTU is for determining phytase activity in feed and premix. Phytase activity may also be determined using the assays of Example 1 (“Determination of phosphatase activity” or “Determination of phytase activity”).

In a particular embodiment the phytase of the invention is isolated. The term “isolated” as used herein refers to a polypeptide which is at least 20% pure, preferably at least 40% pure, more preferably at least 60% pure, even more preferably at least 80% pure, most preferably at least 90% pure, and even most preferably at least 95% pure, as determined by SDS-PAGE. In particular, it is preferred that the polypeptides are in “essentially pure form”, i.e., that the polypeptide preparation is essentially free of other polypeptide material with which it is natively associated. This can be accomplished, for example, by preparing the polypeptide by means of well-known recombinant methods or by classical purification methods.

The relatedness between two amino acid sequences is described by the parameter “identity”. For purposes of the present invention, the alignment of two amino acid sequences is determined by using the Needle program from the EMBOSS package (emboss.org) version 2.8.0. The Needle program implements the global alignment algorithm described in Needleman and Wunsch, 1970, J. Mol. Biol. 48: 443-453. The substitution matrix used is BLOSUM62, gap opening penalty is 10, and gap extension penalty is 0.5.

The degree of identity between an amino acid sequence of the present invention (“invention sequence”) and the amino acid sequence referred to in the claims (SEQ ID NO: 2) is calculated as the number of exact matches in an alignment of the two sequences, divided by the length of the “invention sequence,” or the length of the SEQ ID NO: 2, whichever is the shortest. The result is expressed in percent identity.

An exact match occurs when the “invention sequence” and SEQ ID NO: 2 have identical amino acid residues in the same positions of the overlap (in the alignment example below this is represented by “|”). The length of a sequence is the number of amino acid residues in the sequence (e.g., the length of amino acids 1-411 of SEQ ID NO: 2 is 411).

In a purely hypothetical, alignment example below, the overlap is the amino acid sequence “HTWGER-NL” of Sequence 1; or the amino acid sequence “HGWGEDANL” of Sequence 2. In the example a gap is indicated by a “-”.

Hypothetical Alignment Example:

Sequence 1: ACMSHTWGER-NL
| ||| ||
Sequence 2: HGWGEDANLAMNPS

In a particular embodiment, the percentage of identity of an amino acid sequence of a polypeptide with, or to, SEQ ID NO: 2 is determined by i) aligning the two amino acid sequences using the Needle program, with the BLOSUM62 substitution matrix, a gap opening penalty of 10, and a gap extension penalty of 0.5; ii) counting the number of exact matches in the alignment; iii) dividing the number of exact matches by the length of the shortest of the two amino acid sequences, and iv) converting the result of the division of iii) into percentage.

In the above hypothetical example, the number of exact matches is 6, the length of the shortest one of the two amino acid sequences is 12; accordingly the percentage of identity is 50%.

In particular embodiments of the phytase of the invention, the degree of identity to SEQ ID NO: 2, SEQ ID NO: 4 and/or SEQ ID NO: 6 is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%. In still further particular embodiments, the degree of identity is at least 98.0%, 98.2%, 98.4%, 98.6%, 98.8%, 99.0%, 99.1%, 99.2%, 99.3%, 99.4%, 99.5%, 99.6%, 99.7%, 99.8%, or at least 99.9%. In alternative embodiments, the degree of identity is at least 70%, 71%, 72%, or at least 73%.

In still further particular embodiments, the phytase of the invention has no more than 2, 3, 4, 5, 6, 7, 8, 9, or no more than 10 modifications as compared to SEQ ID NO: 2 SEQ ID NO: 4 and/or SEQ ID NO: 6 or any other parent phytase; no more than 11, 12, 13, 14, 15, 16, 17, 18, 19, or no more than 20 modifications as compared to SEQ ID NO: 2; no more than 21, 22, 23, 24, 25, 26, 27, 28, 29, or no more than 30 modifications as compared to SEQ ID NO: 2; no more than 31, 32, 33, 34, 35, 36, 37, 38, 39, or not more than 40 modifications as compared to SEQ ID NO: 2, SEQ ID NO: 4 and/or SEQ ID NO: 6 or any other parent phytase; no more than 41, 42, 43, 44, 45, 46, 47, 48, 49, or no more than 50 modifications as compared to SEQ ID NO: 2, SEQ ID NO: 4 and/or SEQ ID NO: 6 or any other parent phytase; no more than 51, 52, 53, 54, 55, 56, 57, 58, 59, or no more than 60 modifications as compared to SEQ ID NO: 2, SEQ ID NO: 4 and/or SEQ ID NO: 6 or any other parent phytase; no more than 61, 62, 63, 64, 65, 66, 67, 68, 69, or no more than 70 modifications as compared to SEQ ID NO: 2, SEQ ID NO: 4 and/or SEQ ID NO: 6 or any other parent phytase; no more than 71, 72, 73, 74, 75, 76, 77, 78, 79, or no more than 80 modifications as compared to SEQ ID NO: 2, SEQ ID NO: 4 and/or SEQ ID NO: 6 or any other parent phytase; no more than 81, 82, 83, 84, 85, 86, 87, 88, 89, or no more than 90 modifications as compared to SEQ ID NO: 2, SEQ ID NO: 4 and/or SEQ ID NO: 6 or any other parent phytase; no more than 91, 92, 93, 94, 95, 96, 97, 98, 99, or no more than 100 modifications as compared to SEQ ID NO: 2, SEQ ID NO: 4 and/or SEQ ID NO: 6 or any other parent phytase; no more than 101, 102, 103, 104, 105, 106, 107, 108, 109, or no more than 110 modifications as compared to SEQ ID NO: 2, SEQ ID NO: 4 and/or SEQ ID NO: 6 or any other parent phytase; no more than 111, 112, 113, 114, 115, 116, 117, 118, 119, or no more than 120 modifications as compared to SEQ ID NO: 2, SEQ ID NO: 4 and/or SEQ ID NO: 6 or any other parent phytase; or no more than 121, 122, 123, or 124 modifications as compared to SEQ ID NO: 2, SEQ ID NO: 4 and/or SEQ ID NO: 6 or any other parent phytase.

### Position Numbering

The nomenclature used herein for defining amino acid positions is based on the amino acid sequence of the mature phytase of E. coli deposited in UNIPROT under the id. A7ZK83, which is given in the sequence listing as SEQ ID NO: 2 (amino acids 1-412 of SEQ ID NO: 2). Accordingly, in the present context, the basis for numbering positions is SEQ ID NO: 2 starting with Q1 and ending with S412. (SEQ ID NO: 2) as the standard for position numbering and, thereby, also for the nomenclature.

When used herein the term “mature” part (or sequence) refers to that part of the polypeptide which is secreted by a cell which contains, as part of its genetic equipment, a polynucleotide encoding the polypeptide. In other words, the mature polypeptide part refers to that part of the polypeptide which remains after the signal peptide part, as well as a propeptide part, if any, has been cleaved off. The signal peptide part can be predicted by programs known in the art (e.g., SignalP). Generally, the first amino acid of the mature part of an enzyme can be determined by N-terminal sequencing of the purified enzyme. Any difference between the signal peptide part and the mature part must then be due to to the presence of a propeptide.

Modifications, such as Substitutions, Deletions, Insertions

A phytase variant can comprise various types of modifications relative to a template (i.e., a reference or comparative amino acid sequence such as SEQ ID NO: 2): An amino acid can be substituted with another amino acid; an amino acid can be deleted; an amino acid can be inserted; as well as any combination of any number of such modifications. In the present context the term “insertion” is intended to cover also N- and/or C-terminal extensions.

The general nomenclature used herein for a single modification is the following: XDcY, where “X” and “Y” independently designate a one-letter amino acid code, or a “*” (deletion of an amino acid), “D” designates a number, and “c” designates an alphabetical counter (a, b, c, and so forth), which is only present in insertions. Reference is made to Table 1 below which describes purely hypothetical examples of applying this nomenclature to various types of modifications.

TABLE 1

| | Descrip- | |
|---|---|---|
| Type | tion | Example |
| Substi- | X = Amino | G80A |
| tution | acid in | 80 |
| | template | AALNNSIGVLGVAPSAELYAVKVLGASGSG |
| | D = Posi- | \|\|\|\|\|\|\|:\|\|\|\|\|\|\|\|\|\|\|\|\|\|\|\|\|\|\|\|\|\| |
| | tion in | AALNNSIAVLGVAPSAELYAVKVLGASGSG |
| | template | |
| | c empty | |
| | Y = Amino | |
| | acid in | |
| | variant | |
| Inser- | X = ″*″ | *80aT *80bY *85aS |
| tion | D = Posi- | 80 85 |
| | tion in | AALNNSIG..VLGVA.PSAELYAVKVLGASG |
| | template | \|\|\|\|\|\|\|\| \|\|\|\|\| \|\|\|\|\|\|\|\|\|\|\|\|\|\|\| |
| | before the | AALNNSIGTYVLGVASPSAELYAVKVLGASG |
| | insertion | |
| | c = ″a″ | |
| | for first | |
| | insertion | |
| | at this | |
| | position, | |
| | ″b″ for | |
| | ext, etc | |
| Dele- | X = Amino | V81* |
| tion | acid in | 80 |
| | template | AALNNSIGVLGVAPSAELYAVKVLGASGSG |
| | D = Posi- | \|\|\|\|\|\|\|\| \|\|\|\|\|\|\|\|\|\|\|\|\|\|\|\|\|\|\|\|\| |
| | tion in | AALNNSIG.LGVAPSAELYAVKVLGASGSG |
| | template | |
| | c empty | |
| | Y = ″*″ | |
| N-ter- | Insertions | *0aA *0bT *0cG |
| minal | at posi- | 1 |
| exten- | tion ″0″. | ...AQSVPWGISRVQ |
| sion | | \|\|\|\|\|\|\|\|\|\|\|\| |
| | | ATGAQSVPWGISRVQ |
| C-ter- | Insertions | *275aS *275bT |
| minal | after the | 270 275 |
| exten- | N-termi- | ATSLGSTNLYGSGLVNAEAATR.. |
| sion | nal amino | \|\|\|\|\|\|\|\|\|\|\|\|\|\|\|\|\|\|\|\|\|\| |
| | acid. | ATSLGSTNLYGSGLVNAEAATRST |

As explained above, the position number (“D”) is counted from the first amino acid residue of SEQ ID NO: 2.

Several modifications in the same sequence are separated by “I” (slash), e.g., the designation “1*/2*/3*” means that the amino acids in position number 1, 2, and 3 are all deleted, and the designation “104A/105F” means that the amino acid in position number 104 is substituted by A, and the amino acid in position number 105 is substituted by F.

Alternative modifications are separated by “,” (comma), e.g., the designation “119R,K” means that the amino acid in position 119 is substituted with R or K.

The commas used herein in various other enumerations of possibilities mean what they usually do grammatically, viz. often and/or. E.g., the first comma in the listing “53V,Q, 121D, and/or 167Q” denotes an alternative (V or Q), whereas the two next commas should be interpreted as and/or options: 53 V or Q, and/or 121D, and/or 167Q.

In the present context, “at least one” (e.g., modification) means one or more, e.g., 1, 2, 3, 4, 5, 6, 7, 8, 9, or 10 modifications; or 12, 14, 15, 16, 18, 20, 22, 24, 25, 28, or 30 modifications; and so on, up to a maximum number of modifications of 125, 130, 140, 150, 160, 170, 180, 190, or of 200. The phytase variants of the invention, however, still have to be at least 70% identical to SEQ ID NO: 2, this percentage being determined as described above.

A substitution or extension without any indication of what to substitute or extend with refers to the insertion of any natural, or non-natural, amino acid, except the one that occupies this position in the template.

### Identifying Corresponding Position Numbers

As explained above, the mature the amino acid sequence of the mature E. coli phytase deposited in UNIPROT under the id. A7ZK83 (SEQ ID NO: 2) is used as the standard for position numbering and, thereby, also for the nomenclature.

For another phytase, in particular a phytase variant of the invention, the position corresponding to position D in SEQ ID NO: 2 is found by aligning the two sequences as specified above in the section entitled “Phytase polypeptides, percentage of identity”. From the alignment, the position in the sequence of the invention corresponding to position D of SEQ ID NO: 2 can be clearly and unambiguously identified (the two positions on top of each other in the alignment).

Below some additional, purely hypothetical, examples are included which are derived from Table 1 above which in the third column includes a number of alignments of two sequences:

Consider the third cell in the first row of Table 1: The upper sequence is the template, the lower the variant. Position number 80 refers to amino acid residue G in the template. Amino acid A occupies the corresponding position in the variant. Accordingly, this substitution is designated G80A.

Consider now the third cell in the second row of Table 1: The upper sequence is again the template and the lower the variant. Position number 80 again refers to amino acid residue G in the template. The variant has two insertions, viz. TY, after G80 and before V81 in the template. Whereas the T and Y of course would have their own “real” position number in the variant amino acid sequence, for the present purposes we always refer to the template position numbers, and accordingly the T and the Y are said to be in position number 80a and 80b, respectively.

Finally, consider the third cell in the last row of Table 1: Position number 275 refers to the last amino acid of the template. A C-terminal extension of ST are said to be in position number 275a and 275b, respectively, although, again, of course they have their own “real” position number in the variant amino acid sequence.

### Modified Properties, Reference Phytase

In a particular embodiment, the phytase of the invention has modified, preferably improved, properties. The terms “modified” and “improved” imply a comparison with another phytase. Examples of such other, reference, or comparative, phytases are: SEQ ID NO: 3, SEQ ID NO: 4, SEQ ID NO: 5, and/or SEQ ID NO: 6.

Non-limiting examples of properties that are modified, preferably improved, are the following: Thermostability, pH profile, specific activity, performance in animal feed, protease-sensibility, and/or glycosylation pattern. The phytase of the invention may also have an modified, preferably improved, temperature profile, and/or it may incorporate a change of a potential protease cleavage site.

### Thermal Performance

### Thermostability

Thermostability may be determined as described in Example 3, i.e., using DSC measurements to determine the denaturation temperature, Td, of the purified phytase protein. The Td is indicative of the thermostability of the protein: The higher the Td, the higher the thermostability. Accordingly, in a preferred embodiment, the phytase of the invention has a Td which is higher than the Td of a reference phytase, wherein Td is determined on purified phytase samples (preferably with a purity of at least 90% or 95%, determined by SDS-PAGE).

Thermostability may also be determined as follows. Accordingly, in a preferred embodiment the phytase of the invention, after incubation for 60 minutes at 70° C. and pH 4.0, has an improved residual activity as compared to the residual activity of a reference phytase treated in the same way, the residual activity being calculated for each phytase relative to the activity found before the incubation (at 0 minutes). The residual activity is preferably measured on sodium phytate at pH 5.5 and 37° C. The incubation is preferably in 0.1 M sodium acetate, pH 4.0. The phytase is preferably purified, more preferably to a purity of at least 95%, determined by SDS-PAGE. A preferred phytase activity assay buffer is 0.25 M Na-acetate pH 5.5. Using this method, the residual activity of the phytase of the invention is preferably at least 105% of the residual activity of the reference phytase, more preferably at least 110%, 115%, 120%, 130%, 140%, 150%, 160%, 170%, 180%, 190%, or 200%. In the alternative, the residual activity relative to the activity at 0 minutes is preferably at least 31%, or at least 32%. The following substitutions providing improved thermostability stability are preferred (see Table 9): 273L, 46E, 362R, and/or 53V.

In a particular embodiment, the phytase variant of the invention is more thermostable than the reference phytase, wherein thermostability is determined using any of the above-mentioned four tests (based on the Examples).

### Heat-Stability

Heat stability may be determined as described in Example 4 by determining the temperature/activity profile of the variant phytases.

### Temperature Profile/Temperature Stability

Whether or not a phytase of the invention has a modified temperature profile as compared to a reference phytase may be determined as described in Example 4. Accordingly, in a particular embodiment the phytase of the invention has a modified temperature profile as compared to a reference phytase, wherein the temperature profile is determined as phytase activity as a function of temperature on sodium phytate at pH 5.5 in the temperature range of 20-90° C. (in 10° C. steps). A preferred buffer is in 0.25 M Na-acetate buffer pH 5.5. The activity at each temperature is preferably indicated as relative activity (in %) normalized to the value at optimum temperature. The optimum temperature is that temperature within the tested temperatures (i.e., those with 5-10° C. jumps) where the activity is highest.

### pH Profile

Whether or not a phytase of the invention has a modified pH profile as compared to a reference phytase may be determined as described in the Examples. Accordingly, in a particular embodiment the phytase of the invention has an modified pH profile as compared to a reference phytase, wherein the pH profile is determined as phytase activity as a function of pH on sodium phytate at 37° C. in the pH range of 2.0 to 7.5 (in 0.5 pH-unit steps). A preferred buffer is a cocktail of 50 mM glycine, 50 mM acetic acid and 50 mM Bis-Tris. Another preferred buffer is 0.25 M sodium acetate. The activity at each pH is preferably indicated as relative activity (in %) normalized to the value at optimum pH.

An example of a modified pH profile is where the pH curve (relative activity as a function of pH) is shifted towards higher, or lower, pH.

Another example of an modified pH profile is where the optimum pH is changed, in the upward or the downward direction.

A modified pH profile may also be determined by comparing phosphatase activity at pH 3.5 and 5.5. Alternatively, the activity at pH 3.5 may be compared with the activity at pH 4.0, 4.5, or 5.0. In a still further alternative embodiment, phytase activities are compared instead of phosphatase activities.

In a particular embodiment, the phytase of the invention has an modified pH profile as compared to a reference phytase. More in particular, the pH profile is modified in the pH-range of 3.5-5.5. Still more in particular, the activity at pH 4.0, 4.5, 5.0, and/or 5.5 is at a level of at least 50%, 55%, 60%, 65%, 70%, 75%, 80%, 85%, 90%, or 95% of the activity at the pH-optimum.

### Specific Activity

In a particular embodiment, the phytase of the invention has an improved specific activity relative to a reference phytase. More in particular, the specific activity of a phytase of the invention is at least 105%, relative to the specific activity of a reference phytase determined by the same procedure. In still further particular embodiments, the relative specific activity is at least 110, 115, 120, 125, 130, 140, 145, 150, 160, 170, 180, 190, 200, 220, 240, 260, 280, 300, 350 or even 400%, still relative to the specific activity of the reference phytase as determined by the same procedure.

In the alternative, the term high specific activity refers to a specific activity of at least 200 FYT/mg Enzyme Protein (EP). In particular embodiments, the specific activity is at least 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900 or 3000 FYT/mg EP.

Specific activity is measured on highly purified samples (an SDS poly acryl amide gel should show the presence of only one component). The enzyme protein concentration may be determined by amino acid analysis, and the phytase activity in the units of FYT, determined as described in the Examples. Specific activity is a characteristic of the specific phytase variant in question, and it is calculated as the phytase activity measured in FYT units per mg phytase variant enzyme protein.

### Performance in Animal Feed

In a particular embodiment the phytase of the invention has an improved performance in animal feed as compared to a reference phytase. The performance in animal feed may be determined by an in vitro model of Example 6. Accordingly, in a preferred embodiment the phytase of the invention has an improved performance in animal feed, wherein the performance is determined in an in vitro model, by preparing feed samples composed of 30% soybean meal and 70% maize meal with added CaCl₂ to a concentration of 5 g calcium per kg feed; pre-incubating them at 40° C. and pH 3.0 for 30 minutes followed by addition of pepsin (3000 U/g feed) and phytase; incubating the samples at 40° C. and pH 3.0 for 60 minutes followed by pH 4.0 for 30 minutes; stopping the reactions; extracting phytic acid and inositol-phosphates by addition of HCl to a final concentration of 0.5 M and incubation at 40° C. for 2 hours, followed by one freeze-thaw cycle and 1 hour incubation at 40° C.; separating phytic acid and inositol-phosphates by high performance ion chromatography; determining the amount of residual phytate phosphorus (IP6-P); calculating the difference in residual IP6-P between the phytase-treated and a non-phytase-treated blank sample (this difference is degraded IP6-P); and expressing the degraded IP6-P of the phytase of the invention relative to degraded IP6-P of the reference phytase (e.g., the phytases having SEQ ID NOs: 3 and 4).

The phytase of the invention and the reference phytase are of course dosed in the same amount, preferably based on phytase activity units (FYT). A preferred dosage is 125 FYT/kg feed. Another preferred dosage is 250 FYT/kg feed. The phytases may be dosed in the form of purified phytases, or in the form of fermentation supernatants. Purified phytases preferably have a purity of at least 95%, as determined by SDS-PAGE.

In preferred embodiments, the degraded IP6-P value of the purified phytase of the invention, relative to the degraded IP6-P value of the reference phytase, is at least 101%, or at least 102%, 103%, 104%, 105%, 110%, 115%, or 120%. In still further preferred embodiments, the degraded IP6-P value of the purified phytase of the invention, relative to the degraded IP6-P value of the reference phytase, is at least 125%, 130%, 140%, 150%, 160%, 170%, 180%, 190%, or 200%. Preferably, the degraded IP6-P value of the phytase of the invention, relative to the degraded IP6-P value of the SEQ ID NO: 2 phytase, is at least 105%, 110%, 113%, 115%, 120%, 125%, or 130%.

The relative performance of a phytase of the invention may also be calculated as the percentage of the phosphorous released by the reference phytase.

In a still further particular embodiment, the relative performance of the phytase of the invention may be calculated as the percentage of the phosphorous released by the phytase of the invention, relative to the amount of phosphorous released by the reference phytase.

In still further particular embodiments, the relative performance of the phytase of the invention is at least 105%, preferably at least 110, 120, 130, 140, 150, 160, 170, 180, 190, or 200%.

### Nucleic Acid Sequences and Constructs

The present invention also relates to nucleic acid sequences comprising a nucleic acid sequence which encodes a phytase variant of the invention.

The term “isolated nucleic acid sequence” refers to a nucleic acid sequence which is essentially free of other nucleic acid sequences, e.g., at least about 20% pure, preferably at least about 40% pure, more preferably at least about 60% pure, even more preferably at least about 80% pure, and most preferably at least about 90% pure as determined by agarose electrophoresis. For example, an isolated nucleic acid sequence can be obtained by standard cloning procedures used in genetic engineering to relocate the nucleic acid sequence from its natural location to a different site where it will be reproduced. The cloning procedures may involve excision and isolation of a desired nucleic acid fragment comprising the nucleic acid sequence encoding the polypeptide, insertion of the fragment into a vector molecule, and incorporation of the recombinant vector into a host cell where multiple copies or clones of the nucleic acid sequence will be replicated. The nucleic acid sequence may be of genomic, cDNA, RNA, semisynthetic, synthetic origin, or any combinations thereof.

The nucleic acid sequences of the invention can be prepared by introducing at least one mutation into a template phytase coding sequence or a subsequence thereof, wherein the mutant nucleic acid sequence encodes a variant phytase. The introduction of a mutation into the nucleic acid sequence to exchange one nucleotide for another nucleotide may be accomplished by any of the methods known in the art, e.g., by site-directed mutagenesis, by random mutagenesis, or by doped, spiked, or localized random mutagenesis.

Random mutagenesis is suitably performed either as localized or region-specific random mutagenesis in at least three parts of the gene translating to the amino acid sequence shown in question, or within the whole gene. When the mutagenesis is performed by the use of an oligonucleotide, the oligonucleotide may be doped or spiked with the three non-parent nucleotides during the synthesis of the oligonucleotide at the positions which are to be changed. The doping or spiking may be performed so that codons for unwanted amino acids are avoided. The doped or spiked oligonucleotide can be incorporated into the DNA encoding the phytase enzyme by any technique, using, e.g., PCR, LCR or any DNA polymerase and ligase as deemed appropriate.

Preferably, the doping is carried out using “constant random doping”, in which the percentage of wild-type and mutation in each position is predefined. Furthermore, the doping may be directed toward a preference for the introduction of certain nucleotides, and thereby a preference for the introduction of one or more specific amino acid residues. The doping may be made, e.g., so as to allow for the introduction of 90% wild-type and 10% mutations in each position. An additional consideration in the choice of a doping scheme is based on genetic as well as protein-structural constraints.

The random mutagenesis may be advantageously localized to a part of the parent phytase in question. This may, e.g., be advantageous when certain regions of the enzyme have been identified to be of particular importance for a given property of the enzyme.

Alternative methods for providing variants of the invention include gene shuffling, e.g., as described in WO 95/22625 or in WO 96/00343, and the consensus derivation process as described in EP 897985.

### Nucleic Acid Constructs

A nucleic acid construct comprises a nucleic acid sequence of the present invention operably linked to one or more control sequences which direct the expression of the coding sequence in a suitable host cell under conditions compatible with the control sequences. Expression will be understood to include any step involved in the production of the polypeptide including, but not limited to, transcription, post-transcriptional modification, translation, post-translational modification, and secretion.

The term “nucleic acid construct” as used herein refers to a nucleic acid molecule, either single- or double-stranded, which is isolated from a naturally occurring gene or which is modified to contain segments of nucleic acids in a manner that would not otherwise exist in nature. The term nucleic acid construct is synonymous with the term “expression cassette” when the nucleic acid construct contains the control sequences required for expression of a coding sequence of the present invention.

The term “control sequences” is defined herein to include all components, which are necessary or advantageous for the expression of a polynucleotide encoding a polypeptide of the present invention. Each control sequence may be native or foreign to the nucleotide sequence encoding the polypeptide. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleotide sequence encoding a polypeptide.

The term “operably linked” denotes herein a configuration in which a control sequence is placed at an appropriate position relative to the coding sequence of the polynucleotide sequence such that the control sequence directs the expression of the coding sequence of a polypeptide.

When used herein the term “coding sequence” (CDS) means a nucleotide sequence, which directly specifies the amino acid sequence of its protein product. The boundaries of the coding sequence are generally determined by an open reading frame, which usually begins with the ATG start codon or alternative start codons such as GTG and TTG. The coding sequence may a DNA, cDNA, or recombinant nucleotide sequence.

### Expression Vector

The term “expression” includes any step involved in the production of the polypeptide including, but not limited to, transcription, post-transcriptional modification, translation, post-translational modification, and secretion.

The term “expression vector” is defined herein as a linear or circular DNA molecule that comprises a polynucleotide encoding a polypeptide of the invention, and which is operably linked to additional nucleotides that provide for its expression.

A nucleic acid sequence encoding a phytase variant of the invention can be expressed using an expression vector which typically includes control sequences encoding a promoter, operator, ribosome binding site, translation initiation signal, and, optionally, a repressor gene or various activator genes.

The recombinant expression vector carrying the DNA sequence encoding a phytase variant of the invention may be any vector which may conveniently be subjected to recombinant DNA procedures, and the choice of vector will often depend on the host cell into which it is to be introduced. The vector may be one which, when introduced into a host cell, is integrated into the host cell genome and replicated together with the chromosome(s) into which it has been integrated.

The phytase variant may also be co-expressed together with at least one other enzyme of animal feed interest, such as a phytase, phosphatase, xylanase, galactanase, alpha-galactosidase, protease, phospholipase, amylase, and/or beta-glucanase. The enzymes may be co-expressed from different vectors, from one vector, or using a mixture of both techniques. When using different vectors, the vectors may have different selectable markers, and different origins of replication. When using only one vector, the genes can be expressed from one or more promoters. If cloned under the regulation of one promoter (di- or multi-cistronic), the order in which the genes are cloned may affect the expression levels of the proteins. The phytase variant may also be expressed as a fusion protein, i.e., that the gene encoding the phytase variant has been fused in frame to the gene encoding another protein. This protein may be another enzyme or a functional domain from another enzyme.

### Host Cells

The term “host cell”, as used herein, includes any cell type which is susceptible to transformation, transfection, transduction, and the like with a nucleic acid construct comprising a polynucleotide of the present invention.

The present invention also relates to recombinant host cells, comprising a polynucleotide of the present invention, which are advantageously used in the recombinant production of the polypeptides. A vector comprising a polynucleotide of the present invention is introduced into a host cell so that the vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be a unicellular microorganism, e.g., a prokaryote, or a non-unicellular microorganism, e.g., a eukaryote.

Useful unicellular microorganisms are bacterial cells such as gram positive bacteria including, but not limited to, a Bacillus cell, e.g., Bacillus alkalophilus, Bacillus amyloliquefaciens, Bacillus brevis, Bacillus circulans, Bacillus clausii, Bacillus coagulans, Bacillus lautus, Bacillus lentus, Bacillus licheniformis, Bacillus megaterium, Bacillus stearothermophilus, Bacillus subtilis, and Bacillus thuringiensis; or a Streptomyces cell, e.g., Streptomyces lividans and Streptomyces murinus, or gram negative bacteria such as E. coli and Pseudomonas sp. In a preferred aspect, the bacterial host cell is a Bacillus lentus, Bacillus licheniformis, Bacillus stearothermophilus, or Bacillus subtilis cell. In another preferred aspect, the Bacillus cell is an alkalophilic Bacillus.

The introduction of a vector into a bacterial host cell may, for instance, be effected by protoplast transformation (see, e.g., Chang and Cohen, 1979, Molecular General Genetics 168: 111-115), using competent cells (see, e.g., Young and Spizizin, 1961, Journal of Bacteriology 81: 823-829, or Dubnau and Davidoff-Abelson, 1971, Journal of Molecular Biology 56: 209-221), electroporation (see, e.g., Shigekawa and Dower, 1988, Biotechniques 6: 742-751), or conjugation (see, e.g., Koehler and Thorne, 1987, Journal of Bacteriology 169: 5771-5278).

The host cell may also be a eukaryote, such as a mammalian, insect, plant, or fungal cell.

In a preferred aspect, the host cell is a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra).

In a more preferred aspect, the fungal host cell is a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No. 9, 1980).

In an even more preferred aspect, the yeast host cell is a Candida, Hansenula, Kluyveromyces, Pichia, Saccharomyces, Schizosaccharomyces, or Yarrowia cell.

In a most preferred aspect, the yeast host cell is a Pichia pastoris, Pichia methanolica, Saccharomyces carlsbergensis, Saccharomyces cerevisiae, Saccharomyces diastaticus, Saccharomyces douglasii, Saccharomyces kluyveri, Saccharomyces norbensis or Saccharomyces oviformis cell. In another most preferred aspect, the yeast host cell is a Kluyveromyces lactis cell. In another most preferred aspect, the yeast host cell is a Yarrowia lipolytica cell.

In another more preferred aspect, the fungal host cell is a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

In an even more preferred aspect, the filamentous fungal host cell is an Acremonium, Aspergillus, Aureobasidium, Bjerkandera, Ceriporiopsis, Coprinus, Coriolus, Cryptococcus, Filobasidium, Fusarium, Humicola, Magnaporthe, Mucor, Myceliophthora, Neocallimastix, Neurospora, Paecilomyces, Penicillium, Phanerochaete, Phiebia, Piromyces, Pleurotus, Schizophyllum, Talaromyces, Thermoascus, Thielavia, Tolypocladium, Trametes, or Trichoderma cell.

In a most preferred aspect, the filamentous fungal host cell is an Aspergillus awamori, Aspergillus fumigatus, Aspergillus foetidus, Aspergillus japonicus, Aspergillus nidulans, Aspergillus niger or Aspergillus oryzae cell. In another most preferred aspect, the filamentous fungal host cell is a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium suiphureum, Fusarium torulosum, Fusarium trichothecioides, or Fusarium venenatum cell. In another most preferred aspect, the filamentous fungal host cell is a Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Coprinus cinereus, Coriolus hirsutus, Humicola insolens, Humicola lanuginosa, Mucor miehei, Myceliophthora thermophila, Neurospora crassa, Penicillium purpurogenum, Phanerochaete chrysosporium, Phiebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride strain cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238 023 and Yelton et al., 1984, Proceedings of the National Academy of Sciences USA 81: 1470-1474. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920.

### Methods of Production

The present invention also relates to methods for producing a phytase of the present invention comprising (a) cultivating a host cell under conditions conducive for production of the phytase; and (b) recovering the phytase.

In the production methods of the present invention, the cells are cultivated in a nutrient medium suitable for production of the polypeptide using methods well known in the art. For example, the cell may be cultivated by shake flask cultivation, and small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The resulting polypeptide may be recovered using methods known in the art. For example, the polypeptide may be recovered from the nutrient medium by conventional procedures including, but not limited to, centrifugation, filtration, extraction, spray-drying, evaporation, or precipitation.

The polypeptides of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989).

### Transgenic Plants

The present invention also relates to a transgenic plant, plant part, or plant cell which has been transformed with a nucleotide sequence encoding a polypeptide having phytase activity of the present invention so as to express and produce the polypeptide in recoverable quantities. The polypeptide may be recovered from the plant or plant part. Alternatively, the plant or plant part containing the recombinant polypeptide may be used as such for improving the quality of a food or feed, e.g., improving nutritional value, palatability, and rheological properties, or to destroy an antinutritive factor.

In a particular embodiment, the polypeptide is targeted to the endosperm storage vacuoles in seeds. This can be obtained by synthesizing it as a precursor with a suitable signal peptide, see Horvath et al., 2000, PNAS 97(4): 1914-1919.

The transgenic plant can be dicotyledonous (a dicot) or monocotyledonous (a monocot) or engineered variants thereof. Examples of monocot plants are grasses, such as meadow grass (blue grass, Poa), forage grass such as Festuca, Lolium, temperate grass, such as Agrostis, and cereals, e.g., wheat, oats, rye, barley, rice, sorghum, triticale (stabilized hybrid of wheat (Triticum) and rye (Secale), and maize (corn). Examples of dicot plants are tobacco, legumes, such as sunflower (Helianthus), cotton (Gossypium), lupins, potato, sugar beet, pea, bean and soybean, and cruciferous plants (family Brassicaceae), such as cauliflower, rape seed, and the closely related model organism Arabidopsis thaliana. Low-phytate plants as described in, e.g., U.S. Pat. No. 5,689,054 and U.S. Pat. No. 6,111,168 are examples of engineered plants.

Examples of plant parts are stem, callus, leaves, root, fruits, seeds, and tubers, as well as the individual tissues comprising these parts, e.g., epidermis, mesophyll, parenchyma, vascular tissues, meristems. Also specific plant cell compartments, such as chloroplast, apoplast, mitochondria, vacuole, peroxisomes, and cytoplasm are considered to be a plant part. Furthermore, any plant cell, whatever the tissue origin, is considered to be a plant part. Likewise, plant parts such as specific tissues and cells isolated to facilitate the utilisation of the invention are also considered plant parts, e.g., embryos, endosperms, aleurone and seed coats.

Also included within the scope of the present invention are the progeny of such plants, plant parts and plant cells.

The transgenic plant or plant cell expressing a polypeptide of the present invention may be constructed in accordance with methods known in the art. Briefly, the plant or plant cell is constructed by incorporating one or more expression constructs encoding a polypeptide of the present invention into the plant host genome and propagating the resulting modified plant or plant cell into a transgenic plant or plant cell.

Conveniently, the expression construct is a nucleic acid construct which comprises a nucleic acid sequence encoding a polypeptide of the present invention operably linked with appropriate regulatory sequences required for expression of the nucleic acid sequence in the plant or plant part of choice. Furthermore, the expression construct may comprise a selectable marker useful for identifying host cells into which the expression construct has been integrated and DNA sequences necessary for introduction of the construct into the plant in question (the latter depends on the DNA introduction method to be used).

The choice of regulatory sequences, such as promoter and terminator sequences and optionally signal or transit sequences are determined, for example, on the basis of when, where, and how the polypeptide is desired to be expressed. For instance, the expression of the gene encoding a polypeptide of the present invention may be constitutive or inducible, or may be developmental, stage or tissue specific, and the gene product may be targeted to a specific cell compartment, tissue or plant part such as seeds or leaves. Regulatory sequences are, for example, described by Tague et al., 1988, Plant Physiology 86: 506.

For constitutive expression, the following promoters may be used: The 35S-CaMV promoter (Franck et al., 1980, Cell 21: 285-294), the maize ubiquitin 1 (Christensen AH, Sharrock R A and Quail 1992. Maize polyubiquitin genes: structure, thermal perturbation of expression and transcript splicing, and promoter activity following transfer to protoplasts by electroporation), or the rice actin 1 promoter (Plant Mol. Biol. 18, 675-689.; Zhang et al., 1991, Analysis of rice Act1 5′ region activity in transgenic rice plants. Plant Cell 3: 1155-1165). Organ-specific promoters may be, for example, a promoter from storage sink tissues such as seeds, potato tubers, and fruits (Edwards & Coruzzi, 1990, Ann. Rev. Genet. 24: 275-303), or from metabolic sink tissues such as meristems (Ito et al., 1994, Plant Mol. Biol. 24: 863-878), a seed specific promoter such as the glutelin, prolamin, globulin, or albumin promoter from rice (Wu et al., 1998, Plant and Cell Physiology 39: 885-889), a Vicia faba promoter from the legumin B4 and the unknown seed protein gene from Vicia faba (Conrad et al., 1998, Journal of Plant Physiology 152: 708-711), a promoter from a seed oil body protein (Chen et al., 1998, Plant and Cell Physiology 39: 935-941), the storage protein napA promoter from Brassica napus, or any other seed specific promoter known in the art, e.g., as described in WO 91/14772. Furthermore, the promoter may be a leaf specific promoter such as the rbcs promoter from rice or tomato (Kyozuka et al., 1993, Plant Physiology 102: 991-1000, the chlorella virus adenine methyltransferase gene promoter (Mitra and Higgins, 1994, Plant Molecular Biology 26: 85-93), or the aldP gene promoter from rice (Kagaya et al., 1995, Molecular and General Genetics 248: 668-674), or a wound inducible promoter such as the potato pin2 promoter (Xu et al., 1993, Plant Molecular Biology 22: 573-588). Likewise, the promoter may be inducible by abiotic treatments such as temperature, drought or modifications in salinity or inducible by exogenously applied substances that activate the promoter, e.g., ethanol, oestrogens, plant hormones like ethylene, abscisic acid, gibberellic acid, and/or heavy metals.

A promoter enhancer element may also be used to achieve higher expression of the polypeptide in the plant. For instance, the promoter enhancer element may be an intron which is placed between the promoter and the nucleotide sequence encoding a polypeptide of the present invention. For instance, Xu et al., 1993, supra, disclose the use of the first intron of the rice actin 1 gene to enhance expression.

Still further, the codon usage may be optimized for the plant species in question to improve expression (see Horvath et al. referred to above).

The selectable marker gene and any other parts of the expression construct may be chosen from those available in the art.

The nucleic acid construct is incorporated into the plant genome according to conventional techniques known in the art, including Agrobacterium-mediated transformation, virus-mediated transformation, microinjection, particle bombardment, biolistic transformation, and electroporation (Gasser et al., 1990, Science 244: 1293; Potrykus, 1990, Bio/Technology 8: 535; Shimamoto et al., 1989, Nature 338: 274).

Presently, Agrobacterium tumefaciens-mediated gene transfer is the method of choice for generating transgenic dicots (for a review, see Hooykas and Schilperoort, 1992, Plant Molecular Biology 19: 15-38), and it can also be used for transforming monocots, although other transformation methods are more often used for these plants. Presently, the method of choice for generating transgenic monocots, supplementing the Agrobacterium approach, is particle bombardment (microscopic gold or tungsten particles coated with the transforming DNA) of embryonic calli or developing embryos (Christou, 1992, Plant Journal 2: 275-281; Shimamoto, 1994, Current Opinion Biotechnology 5: 158-162; Vasil et al., 1992, Bio/Technology 10: 667-674). An alternative method for transformation of monocots is based on protoplast transformation as described by Omirulleh et al., 1993, Plant Molecular Biology 21: 415-428.

Following transformation, the transformants having incorporated therein the expression construct are selected and regenerated into whole plants according to methods well-known in the art. Often the transformation procedure is designed for the selective elimination of selection genes either during regeneration or in the following generations by using, e.g., co-transformation with two separate T-DNA constructs or site specific excision of the selection gene by a specific recombinase.

The present invention also relates to methods for producing a polypeptide of the present invention comprising (a) cultivating a transgenic plant or a plant cell comprising a nucleic acid sequence encoding a polypeptide having phytase activity of the present invention under conditions conducive for production of the polypeptide; and (b) recovering the polypeptide.

### Compositions and Uses

In still further aspects, the present invention relates to compositions comprising a polypeptide of the present invention, as well as methods of using these.

The polypeptide compositions may be prepared in accordance with methods known in the art and may be in the form of a liquid or a dry composition. For instance, the polypeptide composition may be in the form of granulates or microgranulates. The polypeptide to be included in the composition may be stabilized in accordance with methods known in the art.

The phytase of the invention can be used for degradation, in any industrial context, of, for example, phytate, phytic acid, and/or the mono-, di-, tri-, tetra- and/or penta-phosphates of myo-inositol. It is well known that the phosphate moieties of these compounds chelates divalent and trivalent cations such as metal ions, i.a. the nutritionally essential ions of calcium, iron, zinc and magnesium as well as the trace minerals manganese, copper and molybdenum. Besides, the phytic acid also to a certain extent binds proteins by electrostatic interaction.

Accordingly, preferred uses of the polypeptides of the invention are in animal feed preparations (including human food) or in additives for such preparations.

In a particular embodiment, the polypeptide of the invention can be used for improving the nutritional value of an animal feed. Non-limiting examples of improving the nutritional value of animal feed (including human food), are: Improving feed digestibility; promoting growth of the animal; improving feed utilization; improving bio-availability of proteins; increasing the level of digestible phosphate; improving the release and/or degradation of phytate; improving bio-availability of trace minerals; improving bio-availability of macro minerals; eliminating the need for adding supplemental phosphate, trace minerals, and/or macro minerals; and/or improving egg shell quality. The nutritional value of the feed is therefore increased, and the growth rate and/or weight gain and/or feed conversion (i.e., the weight of ingested feed relative to weight gain) of the animal may be improved.

Furthermore, the polypeptide of the invention can be used for reducing phytate level of manure.

### Animals, Animal Feed, and Animal Feed Additives

The term animal includes all animals, including human beings. Examples of animals are non-ruminants, and ruminants. Ruminant animals include, for example, animals such as sheep, goat, and cattle, e.g., cow such as beef cattle and dairy cows. In a particular embodiment, the animal is a non-ruminant animal. Non-ruminant animals include mono-gastric animals, e.g., pig or swine (including, but not limited to, piglets, growing pigs, and sows); poultry such as turkeys, ducks and chickens (including but not limited to broiler chicks, layers); fish (including but not limited to salmon, trout, tilapia, catfish and carp); and crustaceans (including but not limited to shrimp and prawn).

The term feed or feed composition means any compound, preparation, mixture, or composition suitable for, or intended for intake by an animal.

In the use according to the invention the polypeptide can be fed to the animal before, after, or simultaneously with the diet. The latter is preferred.

In a particular embodiment, the polypeptide, in the form in which it is added to the feed, or when being included in a feed additive, is substantially pure. In a particular embodiment it is well-defined. The term “well-defined” means that the phytase preparation is at least 50% pure as determined by Size-exclusion chromatography (see Example 12 of WO 01/58275). In other particular embodiments the phytase preparation is at least 60, 70, 80, 85, 88, 90, 92, 94, or at least 95% pure as determined by this method.

A substantially pure, and/or well-defined polypeptide preparation is advantageous. For instance, it is much easier to dose correctly to the feed a polypeptide that is essentially free from interfering or contaminating other polypeptides. The term dose correctly refers in particular to the objective of obtaining consistent and constant results, and the capability of optimising dosage based upon the desired effect.

For the use in animal feed, however, the phytase polypeptide of the invention need not be that pure; it may include, e.g., other polypeptides, in which case it could be termed a phytase preparation.

The phytase preparation can be (a) added directly to the feed (or used directly in a treatment process of proteins), or (b) it can be used in the production of one or more intermediate compositions such as feed additives or premixes that is subsequently added to the feed (or used in a treatment process). The degree of purity described above refers to the purity of the original polypeptide preparation, whether used according to (a) or (b) above.

Polypeptide preparations with purities of this order of magnitude are in particular obtainable using recombinant methods of production, whereas they are not so easily obtained and also subject to a much higher batch-to-batch variation when the polypeptide is produced by traditional fermentation methods.

Such polypeptide preparation may of course be mixed with other polypeptides.

The polypeptide can be added to the feed in any form, be it as a relatively pure polypeptide, or in admixture with other components intended for addition to animal feed, i.e., in the form of animal feed additives, such as the so-called pre-mixes for animal feed.

In a further aspect the present invention relates to compositions for use in animal feed, such as animal feed, and animal feed additives, e.g., premixes.

Apart from the polypeptide of the invention, the animal feed additives of the invention contain at least one fat-soluble vitamin, and/or at least one water soluble vitamin, and/or at least one trace mineral. The feed additive may also contain at least one macro mineral.

Further, optional, feed-additive ingredients are colouring agents, e.g., carotenoids such as beta-carotene, astaxanthin, and lutein; aroma compounds; stabilisers; antimicrobial peptides; polyunsaturated fatty acids; reactive oxygen generating species; and/or at least one other polypeptide selected from amongst phytase (EC 3.1.3.8 or 3.1.3.26); phosphatase (EC 3.1.3.1; EC 3.1.3.2; EC 3.1.3.39); xylanase (EC 3.2.1.8); galactanase (EC 3.2.1.89); alpha-galactosidase (EC 3.2.1.22); protease (EC 3.4.-.-), phospholipase A1 (EC 3.1.1.32); phospholipase A2 (EC 3.1.1.4); lysophospholipase (EC 3.1.1.5); phospholipase C (3.1.4.3); phospholipase D (EC 3.1.4.4); amylase such as, for example, alpha-amylase (EC 3.2.1.1); and/or beta-glucanase (EC 3.2.1.4 or EC 3.2.1.6).

In a particular embodiment these other polypeptides are well-defined (as defined above for phytase preparations).

The phytase of the invention may also be combined with other phytases, for example ascomycete phytases such as Aspergillus phytases, for example derived from Aspergillus ficuum, Aspergillus niger, or Aspergillus awamori; or basidiomycete phytases, for example derived from Peniophora lycii, Agrocybe pediades, Trametes pubescens, or Paxillus involutus; or derivatives, fragments or variants thereof which have phytase activity.

Thus, in preferred embodiments of the use in animal feed of the invention, and in preferred embodiments of the animal feed additive and the animal feed of the invention, the phytase of the invention is combined with such phytases.

Examples of antimicrobial peptides (AMP's) are CAP18, Leucocin A, Tritrpticin, Protegrin-1, Thanatin, Defensin, Lactoferrin, Lactoferricin, and Ovispirin such as Novispirin (Robert Lehrer, 2000), Plectasins, and Statins, including the compounds and polypeptides disclosed in WO 03/044049 and WO 03/048148, as well as variants or fragments of the above that retain antimicrobial activity.

Examples of antifungal polypeptides (AFP's) are the Aspergillus giganteus, and Aspergillus niger peptides, as well as variants and fragments thereof which retain antifungal activity, as disclosed in WO 94/01459 and WO 02/090384.

Examples of polyunsaturated fatty acids are C18, C20 and C22 polyunsaturated fatty acids, such as arachidonic acid, docosohexaenoic acid, eicosapentaenoic acid and gamma-linoleic acid.

Examples of reactive oxygen generating species are chemicals such as perborate, persulphate, or percarbonate; and polypeptides such as an oxidase, an oxygenase or a syntethase.

Usually fat- and water-soluble vitamins, as well as trace minerals form part of a so-called premix intended for addition to the feed, whereas macro minerals are usually separately added to the feed. Either of these composition types, when enriched with a polypeptide of the invention, is an animal feed additive of the invention.

In a particular embodiment, the animal feed additive of the invention is intended for being included (or prescribed as having to be included) in animal diets or feed at levels of 0.01 to 10.0%; more particularly 0.05 to 5.0%; or 0.2 to 1.0% (% meaning g additive per 100 g feed). This is so in particular for premixes.

The following are non-exclusive lists of examples of these components:

Examples of fat-soluble vitamins are vitamin A, vitamin D3, vitamin E, and vitamin K, e.g., vitamin K3.

Examples of water-soluble vitamins are vitamin B12, biotin and choline, vitamin B1, vitamin B2, vitamin B6, niacin, folic acid and panthothenate, e.g., Ca-D-panthothenate.

Examples of trace minerals are manganese, zinc, iron, copper, iodine, selenium, and cobalt.

Examples of macro minerals are calcium, phosphorus and sodium.

The nutritional requirements of these components (exemplified with poultry and piglets/pigs) are listed in Table A of WO 01/58275. Nutritional requirement means that these components should be provided in the diet in the concentrations indicated.

In the alternative, the animal feed additive of the invention comprises at least one of the individual components specified in Table A of WO 01/58275. At least one means either of, one or more of, one, or two, or three, or four and so forth up to all thirteen, or up to all fifteen individual components. More specifically, this at least one individual component is included in the additive of the invention in such an amount as to provide an in-feed-concentration within the range indicated in column four, or column five, or column six of Table A.

The present invention also relates to animal feed compositions. Animal feed compositions or diets have a relatively high content of protein. Poultry and pig diets can be characterised as indicated in Table B of WO 01/58275, columns 2-3. Fish diets can be characterised as indicated in column 4 of this Table B. Furthermore such fish diets usually have a crude fat content of 200-310 g/kg.

WO 01/58275 corresponds to U.S. Ser. No. 09/779,334 which is hereby incorporated by reference.

An animal feed composition according to the invention has a crude protein content of 50-800 g/kg, and furthermore comprises at least one polypeptide as claimed herein.

Furthermore, or in the alternative (to the crude protein content indicated above), the animal feed composition of the invention has a content of metabolisable energy of 10-30 MJ/kg; and/or a content of calcium of 0.1-200 g/kg; and/or a content of available phosphorus of 0.1-200 g/kg; and/or a content of methionine of 0.1-100 g/kg; and/or a content of methionine plus cysteine of 0.1-150 g/kg; and/or a content of lysine of 0.5-50 g/kg.

In particular embodiments, the content of metabolisable energy, crude protein, calcium, phosphorus, methionine, methionine plus cysteine, and/or lysine is within any one of ranges 2, 3, 4 or 5 in Table B of WO 01/58275 (R. 2-5).

Crude protein is calculated as nitrogen (N) multiplied by a factor 6.25, i.e., Crude protein (g/kg)=N (g/kg)×6.25. The nitrogen content is determined by the Kjeldahl method (A.O.A.C., 1984, Official Methods of Analysis 14th ed., Association of Official Analytical Chemists, Washington D.C.).

Metabolisable energy can be calculated on the basis of the NRC publication Nutrient requirements in swine, ninth revised edition 1988, subcommittee on swine nutrition, committee on animal nutrition, board of agriculture, national research council. National Academy Press, Washington, D.C., pp. 2-6, and the European Table of Energy Values for Poultry Feed-stuffs, Spelderholt centre for poultry research and extension, 7361 DA Beekbergen, The Netherlands. Grafisch bedrijf Ponsen & looijen by, Wageningen. ISBN 90-71463-12-5.

The dietary content of calcium, available phosphorus and amino acids in complete animal diets is calculated on the basis of feed tables such as Veevoedertabel 1997, gegevens over chemische samenstelling, verteerbaarheid en voederwaarde van voedermiddelen, Central Veevoederbureau, Runderweg 6, 8219 pk Lelystad. ISBN 90-72839-13-7.

In a particular embodiment, the animal feed composition of the invention contains at least one protein. The protein may be an animal protein, such as meat and bone meal, and/or fish meal; or it may be a vegetable protein. The term vegetable proteins as used herein refers to any compound, composition, preparation or mixture that includes at least one protein derived from or originating from a vegetable, including modified proteins and protein-derivatives. In particular embodiments, the protein content of the vegetable proteins is at least 10, 20, 30, 40, 50, or 60% (w/w).

Vegetable proteins may be derived from vegetable protein sources, such as legumes and cereals, for example materials from plants of the families Fabaceae (Leguminosae), Cruciferaceae, Chenopodiaceae, and Poaceae, such as soy bean meal, lupin meal and rapeseed meal.

In a particular embodiment, the vegetable protein source is material from one or more plants of the family Fabaceae, e.g., soybean, lupine, pea, or bean.

In another particular embodiment, the vegetable protein source is material from one or more plants of the family Chenopodiaceae, e.g., beet, sugar beet, spinach or quinoa.

Other examples of vegetable protein sources are rapeseed, sunflower seed, cotton seed, and cabbage.

Soybean is a preferred vegetable protein source.

Other examples of vegetable protein sources are cereals such as barley, wheat, rye, oat, maize (corn), rice, triticale, and sorghum.

In still further particular embodiments, the animal feed composition of the invention contains 0-80% maize; and/or 0-80% sorghum; and/or 0-70% wheat; and/or 0-70% Barley; and/or 0-30% oats; and/or 0-40% soybean meal; and/or 0-25% fish meal; and/or 0-25% meat and bone meal; and/or 0-20% whey.

Animal diets can be be manufactured, e.g., as mash feed (non pelleted) or pelleted feed. Typically, the milled feed-stuffs are mixed and sufficient amounts of essential vitamins and minerals are added according to the specifications for the species in question. Polypeptides can be added as solid or liquid polypeptide formulations. For example, a solid polypeptide formulation is typically added before or during the mixing step; and a liquid polypeptide preparation is typically added after the pelleting step. The polypeptide may also be incorporated in a feed additive or premix.

The final polypeptide concentration in the diet is within the range of 0.01-200 mg polypeptide protein per kg diet, for example in the range of 5-30 mg polypeptide protein per kg animal diet.

The phytase of the invention should of course be applied in an effective amount, i.e., in an amount adequate for improving solubilisation and/or improving nutritional value of feed. It is at present contemplated that the polypeptide is administered in one or more of the following amounts (dosage ranges): 0.01-200; 0.01-100; 0.5-100; 1-50; 5-100; 10-100; 0.05-50; or 0.10-10—all these ranges being in mg phytase polypeptide protein per kg feed (ppm).

For determining mg phytase polypeptide protein per kg feed, the phytase is purified from the feed composition, and the specific activity of the purified phytase is determined using a relevant assay. The phytase activity of the feed composition as such is also determined using the same assay, and on the basis of these two determinations, the dosage in mg phytase protein per kg feed is calculated.

The same principles apply for determining mg phytase polypeptide protein in feed additives. Of course, if a sample is available of the phytase used for preparing the feed additive or the feed, the specific activity is determined from this sample (no need to purify the phytase from the feed composition or the additive).

### Methods for Producing Fermentation Products

Yet another aspect of the present invention relates to the methods for producing a fermentation product, such as, e.g., ethanol, beer, wine, distillers dried grains (DDG), wherein the fermentation is carried out in the presence of a phytase produced by the present invention. Examples of fermentation processes include, for example, the processes described in WO 01/62947. Fermentation is carried out using a fermenting microorganism, such as, yeast.

In a particular embodiment, the present invention provides methods for producing fermentation product, comprising (a) fermenting (using a fermenting microorganism, such as yeast) a carbohydrate containing material (e.g., starch) in the presence of a phytase of the present invention and (b) producing the fermentation product from the fermented carbohydrate containing material.

In a particular embodiment, the present invention provides methods for producing ethanol, comprising fermenting (using a fermenting microorganism, such as yeast) a carbohydrate containing material (e.g., starch) in the presence of a phytase of the present invention and producing or recovering ethanol from the fermented carbohydrate containing material.

In another embodiment, the present invention provides methods for producing ethanol comprising a) hydrolyzing starch, e.g., by a liquefaction and/or saccharification process, a raw starch hydrolysis process, b) fermenting the resulting starch in the presence of a phytase of the present invention, and c) producing ethanol.

The phytase may be added to the fermentation process at any suitable stage and in any suitable composition, including alone or in combination with other enzymes, such as, one or more alpha-amylases, glucoamylases, proteases, and/or cellulases.

In another embodiment, the present invention provides methods for producing ethanol comprising hydrolyzing biomass, and fermenting (using a fermenting microorganism, such as yeast) the resulting biomass in the presence of a phytase of the present invention.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

Various references are cited herein, the disclosures of which are incorporated by reference in their entireties.

### EXAMPLES

Chemicals used are commercial products of at least reagent grade.

### Example 1

### Preparation of Variants, and Determination of Activity

### Preparation of Phytase Variants

Expression of Phytase Variants in Aspergillus oryzae

The constructs comprising the E. coli phytase variant genes in the examples were used to construct expression vectors for Aspergillus. The Aspergillus expression vectors consist of an expression cassette based on the Aspergillus niger neutral amylase II promoter fused to the Aspergillus nidulans triose phosphate isomerase non translated leader sequence (Pna2/tpi) and the Aspergillus niger amyloglycosidase terminator (Tamg). Also present on the plasmid was the Aspergillus selective marker amdS from Aspergillus nidulans enabling growth on media for transformed aspergillus where acetamid is the sole nitrogen source. The expression plasmids for phytase variants were transformed into Aspergillus as described in Lassen et al., 2001, Applied and Environmental Microbiology 67: 4701-4707. For each of the constructs 4-6 strains were isolated, purified and cultivated in microtiterplates. Expression was determined using a p-nitrophenyl phosphate substrate. The best producing strain was fermented in shake flasks.

Purification of E. coli Phytase Variants

The fermentation supernatant with the phytase variant was filtered through a sandwich of four Whatman glass microfibre filters (2.7, 1.6, 1.2 and 0.7 micrometer). Following this the solution was filtered through a Fast PES Bottle top filter with a 0.22 μm cut-off. The solution was added solid ammonium sulfate giving a final concentration of 1.5 M and the pH was adjusted to 6.0. The solution became a little cloudy and this precipitation was removed by filtration through a Fast PES Bottle top filter with a 0.22 μm cut-off.

The phytase-containing solution was applied to a butyl-sepharose column, approximately 50 ml in a XK26 column, using as buffer A 25 mM bis-tris (Bis-(2-hydroxyethyl)imino-tris(hydroxymethyl)methan))+1.5 M ammonium sulfate pH 6.0, and as buffer B 25 mM bis-tris pH 6.0. The fractions from the column were analyzed for activity using the phosphatase assay (see below) and fractions with activity were pooled.

The pooled fractions were dialyzed extensively against 10 mM Na-acatate, pH 4.5. After dialysis a minor precipitation had formed and this was removed by filtration through a Fast PES bottle top filter with a 0.22 micrometer cut-off. The phytase variant was purified by chromatography on SP Sepharose Fast Flow, approximately 50 ml in a XK26 column, using as buffer A 50 mM sodium acetate pH 4.5, and as buffer B 50 mM sodium acetate+1 M NaCl pH 4.5. The fractions from the column were analyzed for activity using the phosphatase assay (see below) and fractions with activity were pooled. The pooled fractions were tested for phytase activity (see below) and found to be active (>200 FYT/ml).

The molecular weight, as estimated from SDS-PAGE, was approximately 50-55 kDa and the purity was >95%.

### Determination of Phosphatase Activity

75 microliter phytase-containing enzyme solution is dispensed in a microtiter plate well, e. g. NUNC 269620 and 75 microliter substrate is added (for preparing the substrate, two 5 mg p-nitrophenyl phosphate tablets (Sigma, Cat. No. N-9389) are dissolved in 10 ml 0.1 M Na-acetate buffer, pH 5.5). The plate is sealed and incubated 15 min., shaken with 750 rpm at 37° C. After the incubation time 75 microliter stop reagent is added (the stop reagent is 0.1 M di-sodiumtetraborate in water) and the absorbance at 405 nm is measured in a microtiter plate spectrophotometer. One phosphatase unit is defined as the enzyme activity that releases 1 micromol phosphate/min under the given reaction conditions (buffer blind subtracted). The absorbance of 1 micromol p-nitrophenol is determined to be 56 AU (AU=absorbancy units) under assay conditions.

### Determination of Phytase Activity

75 microliters phytase-containing enzyme solution, appropriately diluted in 0.25 M sodium acetate, 0.005% (w/v) Tween-20. pH 5.5, is dispensed in a microtiter plate well, e. g. NUNC 269620, and 75 microliter substrate is added (prepared by dissolving 100 mg sodium phytate from rice (Aldrich Cat. No. 274321) in 10 ml 0.25 M sodium acetate buffer, pH 5.5). The plate is sealed and incubated 15 min. shaken with 750 rpm at 37° C. After incubation, 75 microliter stop reagent is added (the stop reagent being prepared by mixing 10 ml molybdate solution (10% (w/v) ammonium hepta-molybdate in 0.25% (w/v) ammonia solution), 10 ml ammonium vanadate (0.24% commercial product from Bie&Berntsen, Cat. No. LAB17650), and 20 ml 21.7% (w/v) nitric acid), and the absorbance at 405 nm is measured in a microtiter plate spectrophotometer. The phytase activity is expressed in the unit of FYT, one FYT being the amount of enzyme that liberates 1 micromole inorganic ortho-phosphate per minute under the conditions above. An absolute value for the measured phytase activity may be obtained by reference to a standard curve prepared from appropriate dilutions of inorganic phosphate, or by reference to a standard curve made from dilutions of a phytase enzyme preparation with known activity (such standard enzyme preparation with a known activity is available on request from Novozymes A/S, Krogshoejvej 36, DK-2880 Bagsvaerd).

### Example 2

### Specific Activity

The specific activity of a phytase variant is determined on highly purified samples dialysed against 250 mM sodium acetate, pH 5.5. The purity is checked beforehand on an SDS poly acryl amide gel showing the presence of only one component.

The protein concentration is determined by amino acid analysis as follows: An aliquot of the sample is hydrolyzed in 6 N HCl, 0.1% phenol for 16 hours at 110° C. in an evacuated glass tube. The resulting amino acids are quantified using an Applied Biosystems 420A amino acid analysis system operated according to the manufacturer's instructions. From the amounts of the amino acids the total mass—and thus also the concentration—of protein in the hydrolyzed aliquot can be calculated.

The phytase activity is determined in the units of FYT as described in Example 1 (“Determination of phytase activity”), and the specific activity is calculated as the phytase activity measured in FYT units per mg phytase variant enzyme protein.

### Example 3

### Thermostability

An aliquot of the protein sample of E. coli phytase variant (purified as described in Example 1) was either, desalted and buffer-changed into 20 mM Na-acetate, pH 4.0 using a prepacked PD-10 column, or dialysed against 2×500 ml 20 mM Na-acetate, pH 4.0 at 4° C. in a 2-3h step followed by an overnight step. The sample was 0.45 μm filtered and diluted with buffer to approx. 2 A280 units. The dialysis buffer was used as reference in Differential Scanning calorimetry (DSC). The samples were degassed using vacuum suction and stirring for approx. 10 minutes.

A DSC scan was performed on a MicroCal VP-DSC at a constant scan rate of 1.5° C./min from 20-90° C. Data-handling was performed using the MicroCal Origin software (version 4.10), and the denaturation temperature, Td (also called the melting temperature, Tm) is defined as the temperature at the apex of the peak in the thermogram.

The results of DSC for E. coli phytase variants are summarized in the Table 2 below.

TABLE 2
Comparative Thermostability of E. coli Phytases

| | | Td 1st |
|---|---|---|
| | | Scan |
| Variant | Modification | (° C.) |
| Ec-phyt01 | A116T | 61.8 |
| Ec-phyt02 | SEQ ID NO: 2 | 57.6 |
| Ec-phyt06 | G52C/A99C = A | 60.1 |
| Ec-phyt07 | D31C/L177C = C | 53.4 |
| Ec-phyt08 | T141C/V200C = B | 58.7 |
| Ec-phyt09 | G52C/A99C/T141C/V200C/D31C/ | 66.1 |
| | L177C/L410LR = A/B/C/L410LR | |

### Example 4

### Temperature Profile

The temperature profile (phytase activity as a function of temperature) was determined for the E. coli phytase and variants in the temperature range of 20-90° C. essentially as described above (“Determination of phytase activity”). However, the enzymatic reactions (100 microliters phytase-containing enzyme solution+100 microliters substrate) were performed in PCR tubes instead of microtiter plates. After a 15 minute reaction period at desired temperature the tubes were cooled to 20° C. for 20 seconds and 150 microliters of each reaction mixture was transferred to a microtiter plate. 75 microliter stop reagent was added and the absorbance at 405 nm was measured in a microtiter plate spectrophotometer. The results are summarized in Table 3 below. The numbers given for each temperature are relative activity (in %) normalized to the value at optimum.

TABLE 3
Relative temperature profiles

| Phytase | Temperature (° C.) | | | | | | | | | | |
|---|---|---|---|---|---|---|---|---|---|---|---|
| variant | 20 | 30 | 40 | 50 | 60 | 65 | 70 | 75 | 80 | 85 | 90 |
| Ec- | 19 | 33 | 53 | 81 | 100 | 86 | 23 | 14 | 12 | 10 | 7 |
| phy01 | | | | | | | | | | | |
| Ec- | 15 | 28 | 45 | 70 | 89 | 100 | 89 | 21 | 14 | 11 | 9 |
| phy06 | | | | | | | | | | | |
| Ec- | 18 | 34 | 54 | 82 | 100 | 83 | 18 | 13 | 11 | 10 | 7 |
| phy07 | | | | | | | | | | | |
| Ec- | 14 | 25 | 38 | 61 | 82 | 100 | 38 | 13 | 10 | 9 | 7 |
| phy08 | | | | | | | | | | | |
| Ec- | 10 | 19 | 33 | 54 | 69 | 95 | 99 | 100 | 16 | 10 | 6 |
| phy09 | | | | | | | | | | | |

### Example 5

### pH Profile

The pH profile was determined at 37° C. in the pH range of 2.0 to 7.5 (in 0.5 pH-unit steps) as described above in the section “Determination of phytase activity”, except that a buffer cocktail (50 mM glycine, 50 mM acetic acid and 50 mM Bis-Tris was used instead of the 0.25 M sodium acetate pH 5.5 buffer. The results are summarized in table 5 below. The values given for each pH in the range of 2.0-7.5 are the relative activity in % normalized to the value at optimum.

TABLE 5
Relative pH profiles at 37° C.

| | pH | | | | | | | | | | | |
|---|---|---|---|---|---|---|---|---|---|---|---|---|
| Mutation | 2 | 2.5 | 3 | 3.5 | 4 | 4.5 | 5 | 5.5 | 6 | 6.5 | 7 | 7.5 |
| Ec-phyt01 | 54 | 67 | 79 | 86 | 96 | 99 | 100 | 91 | 79 | 52 | 16 | 2 |
| Ec-phyt06 | 55 | 67 | 76 | 81 | 92 | 98 | 100 | 94 | 78 | 53 | 16 | 1 |
| Ec-phyt07 | 57 | 72 | 83 | 88 | 98 | 99 | 100 | 89 | 72 | 46 | 14 | 2 |
| Ec-phyt08 | 52 | 65 | 78 | 85 | 96 | 100 | 98 | 88 | 72 | 46 | 13 | 1 |
| Ec-phyt09 | 59 | 71 | 82 | 93 | 96 | 100 | 92 | 77 | 59 | 35 | 9 | 0 |

### Example 6

### Performance in Animal Feed in an In Vitro Model

The performance in animal feed of a number of phytase variants of the invention is compared in an in vitro model to the performance of a reference protein such as SEQ ID NO:2. The in vitro model simulates gastro-intestinal conditions in a monogastric animal and correlates well with results obtained in animal trials in vivo. The version used in this example simulates the crop and stomach of a broiler. The comparison is performed as follows:

Phytase activity in the variant sample is determined as described in Example 1 under “Determination of phytase activity”.

Feed pellets from a broiler feeding trial—and with maize, soybean meal and soybean oil as main constituents—are pre-incubated at 40° C. and pH 4.6 for 5 minutes followed by the addition of suitable dosages of the phytases (identical dosages are used for all phytases to be tested to allow comparison), for example between 125 to 1000 phytase units FYT/kg feed, or buffer in the control samples. After 5 minutes of incubation, pepsin (3000 U/g feed) in an HCl-solution is added and in this way pH is reduced to 3. The samples are then incubated at 40° C. for another 5 minutes.

The reactions are stopped and phytic acid and inositol-phosphates extracted by addition of HCl to a final concentration of 0.5 M and incubation at 40° C. for 2 hours, followed by one freeze-thaw cycle and 1 hour incubation at 40° C.

Phytic acid and inositol-phosphates are separated by high performance ion chroma-tography as described by Chen et al., 2003, Journal of Chromatography A 1018: 41-52 and quantified as described by Skoglund et al., 1997, J. Agric. Food Chem. 45: 431-436.

Degradation of phytate is then calculated as the difference in inositol-6-phosphate bound phosphorous (IP6-P) between phytase-treated and non-treated samples. The relative performance of the variant is calculated as the percentage of phytate degradation by the wild-type phytase.

### Example 7

### Performance in an In Vivo Pig Trial

Comparative Evaluation of the Effects of Graded Amounts of the E. coli Wild-Type Phytase and a Variant on the Faecal Digestibility and Excretion of Phosphorus and Calcium in Growing Pigs.

Sixty four Large White×Landrace pigs having an initial body weight of 43.55±4.35 kg are used.

The animals are housed in floor-pen cages in an environmentally controlled room. Each pen has a plastic-coated welded wire floor and is equipped with two water nipples and four stainless-steel individualized feeders. Room temperature was 21-22° C. and humidity percentage is 50%.

The pigs are fed a basal diet formulated to provide phosphorus (P) exclusively from vegetable origin during an adaptive period of 14 days. After that period they are allocated into 16 equal groups of 4 animals each.

They are fed for 12 days the basal diet or this diet supplemented with 1000 or 2000 U/kg of E. coli wild-type phytase or with 500, 1000 or 2000 U/kg of the variant designated 100 having 2 additional disulfide bonds.

An indigestible tracer (chromium oxide) is added at a concentration of 0.4% to all the diets allowing calculation of the digestibility of P and calcium (Ca). The feed is distributed ad libitum in mash form, under pen feed consumption control, and the animals has free access to drinking water. The digestibility of Ca is not corrected for Ca intake with the drinking water.

Faecal P, Ca and Cr concentrations are measured at the 12^{th} day of the second period. Faeces were sampled individually, in approximately the same amount at the same time of the day, during the last 3 days preceding that date. Thus, for each dietary treatment and for each criterion a total of 12 individual determinations are performed. All minerals are determined according to standard Association of Official Analytical Chemists (1990) methods using a Vista-MPX ICP-OES spectrometer. The apparent digestibility (% of the intake) of the minerals is calculated for the mentioned 3 day period.

1-34. (canceled)

35. A variant phytase having at least 70% identity to SEQ ID NO:2 and comprising a disulfide bridge at one or more position pairs selected from the group consisting of: A) 52C/99C, B) 141C/200C, C) 31C/177C, D) 91C/46C, E) 31C/176C, F) 59C/100C, and G) 162C/248C.

36. The variant phytase of claim 35, which comprises a first disulfide bridge in the position pair A between residues at positions 52 and 99.

37. The variant phytase of claim 35, which comprises a disulfide bridge in the position pair B between residues at positions 141 and 200.

38. The variant phytase of claim 35, which comprises two disulfide bridges at the position pairs selected from the group consisting of:
A+B, A+C, A+D, A+E, A+F, A+G, B+C, B+D, B+E, B+F, B+G, C+D, C+E, C+F, C+G, D+E, D+F, D+G, E+F, E+G, and F+G, wherein A means 52C/99C, B means 141C/200C, C means 31C/177C, D means 91C/46C, E means 31C/176C, F means 59C/100C, and G means 162C/248C.

39. The variant phytase of claim 35, which comprises three disulfide bridges at the position pairs selected from the group consisting of:
A+B+C, A+B+D, A+B+E, A+B+F, A+B+G, A+C+D, A+C+E, A+C+F, A+C+G, A+D+E, A+D+F, A+D+G, A+E+F, A+E+G, A+F+G, B+C+D, B+C+E, B+C+F, B+C+G, B+D+E, B+D+F, B+D+G, B+E+F, B+E+G, B+F+G, C+D+E, C+D+F, C+D+G, C+E+F, C+E+G, C+F+G, D+E+F, D+E+G, D+F+G, and E+F+G, wherein A means 52C/99C, B means 141C/200C, C means 31C/177C, D means 91C/46C, E means 31C/176C, F means 59C/100C, and G means 162C/248C.

40. The variant phytase of claim 35, which comprises four disulfide bridges at the position pairs selected from the group consisting of:
A+B+C+D, A+B+C+E, A+B+C+F, A+B+C+G, A+B+D+E, A+B+D+F, A+B+D+G, A+B+E+F, A+B+E+G, A+B+F+G, A+C+D+E, A+C+D+F, A+C+D+G, A+C+E+F, A+C+E+G, A+C+E+H, A+C+F+G, A+D+E+F, A+D+E+G, A+D+F+G, A+E+F+G, B+C+D+E, B+C+D+F, B+C+D+G, B+C+E+F, B+C+E+G, B+C+F+G, B+D+E+F, B+D+E+G, B+D+F+G, B+E+F+G, C+D+E+F, C+D+E+G, C+D+F+G, C+E+F+G, C+E+F+H, and D+E+F+G, wherein A means 520/990, B means 141C/200C, C means 31C/177C, D means 91C/46C, E means 31C/176C, F means 59C/100C, and G means 162C/248C.

41. The variant phytase of claim 35, which comprises five disulfide bridges at the position pairs selected from the group consisting of:
A+B+C+D+E, A+B+C+D+F, A+B+C+D+G, A+B+C+E+F, A+B+C+E+G, A+B+C+F+G, A+B+D+E+F, A+B+D+E+G, A+B+D+F+G, A+B+E+F+G, A+B+F+G+H, A+C+D+E+F, A+C+D+E+G, A+C+D+F+G, A+C+E+F+G, A+D+E+F+G, B+C+D+E+F, B+C+D+E+G, B+C+D+F+G, B+C+E+F+G, B+D+E+F+G, and C+D+E+F+G, wherein A means 520/990, B means 141C/200C, C means 31C/177C, D means 91C/46C, E means 31C/176C, F means 59C/100C, and G means 162C/248C.

42. The variant phytase of claim 35, which comprises six disulfide bridges at the position pairs selected from the group consisting of:
A+B+C+D+E+F, A+B+C+D+E+G, A+B+C+D+F+G, A+B+C+E+F+G, A+B+D+E+F+G, A+C+D+E+F+G, and B+C+D+E+F+G, wherein A means 520/990, B means 141C/200C, C means 31C/177C, D means 91C/46C, E means 31C/176C, F means 59C/100C, and G means 162C/248C

43. The variant phytase of claim 35, which comprises seven disulfide bridges at the position pairs A+B+C+D+E+F+G, wherein A means 520/990, B means 141C/200C, C means 31C/177C, D means 91C/46C, E means 31C/176C, F means 59C/100C, and G means 162C/248C.

44. The variant phytase of claim 35, wherein the substitutions to establish the disulfide bridges are:
A′. G52C/A99C
B′. T141C/V200C
C′. D31C/L177C
D′. E91C/W46C
E′. D31C/N176C
F′. G59C/F100C
G′. A162C/S248C

45. The variant phytase of claim 35, further comprising a modification selected from the group consisting of:
A25F, W37F, P38Y, C75K, C75V, C75E, C77A, C108A, T114H, P123E, N126Y, P127L, P127V, C133A, N137E, N137V, T141R, D142R, E146R, G157R, P173Y, C178A, C188A, C204N, C204D, V211W, G233E, G235Y, Q253V, R267A, K286F, Q287Y, N317L, W318Y, T327Y, S367F, C382A, C391A, C408A, and/or from the following combinations W37F/P38Y, P123E/P127L, N126Y/P127V, G233E/G235Y, K286F/Q287Y, N317L/W318Y, W37F/P38Y/P123E/P127L, W37F/P38Y/N126Y/P127V, P173Y/N317L/W318Y, C75K/C204N, C75K/C204N/C178A/C188A, C75K/C204N/C382A/C391A, C75K/C204N/C178A/C188A/C382A/C391A, C75K/C204N/C77A/C108A, C75K/C204N/C133A/C108A, A25F/C75V/T114H/N137E/T141R/E146R/G157R/C204D/V211W/Q253V/R267A/T327Y/C178A/C188A, A25F/C75V/T114H/N137E/T141R/E146R/G157R/C204D/V211W/Q253V/R267A/T327Y/C382A/C391A, A25F/C75V/T114H/N137E/T141R/E146R/G157R/C204D/V211W/Q253V/R267A/T327Y/C178A/C188A/C382A/C391A, A25F/C75V/T114H/N137E/T141R/E146R/G157R/C204 D/V211W/Q253V/R267A/T327Y/C77A/C108A, A25F/C75V/T114H/N137E/T141R/E146R/G157R/C204D/V211W/Q253V/R267A/T327Y/C133A/C408A, A25F/C75E/T114H/N137V/G157R/C204D/V211W/Q253V/R267A/T327Y, A25F/C75E/N137V/T141R/D142R/G157R/C204 D/V211W/Q253V/T327Y, A25F/T114H/N137V/T141R/D142R/G157R/C204D/V211W/Q253V/T327Y, A25F/T114H/N137V/T141R/D142R/E146R/G157R/C204D/V211W/Q253V/R267A/T327Y, A25F/C75V/T114H/N137V/T141R/E146R/G157R/C204 D/V211W/Q253V/R267A/T327Y, A25F/C75V/T114H/N137V/T141R/D142R/E146R/G157R/C204D/V211W/Q253V/T327Y, A25F/T114H/N137V/T141R/D142R/E146R/G157R/C204D/V211W/Q253V/T327Y, A25F/C75E/T114H/N137V/T141R/D142R/G157R/C204D/V211W/Q253V/T327Y, A25F/C75E/T114H/N137V/T141R/D142R/E146R/G157R/V211W/Q253V/R267A/T327Y, A25F/C75E/T114H/N137E/T141R/D142R/G157R/V211W/Q253V/R267A/T327Y/L341P, A47F/C97V/T136H/N159V/T163R/D164R/G179R/V233W/Q275V/R289A/T349Y, A25F/C75E/T114H/N137V/T141R/D142R/E146R/G157R/C204D/V211W/Q253V/R267A/T327Y, A25F/C75E/N137V/D142R/G157R/C204D/V211W/Q253V/R267A/T327Y, A25F/C75E/N137E/T141R/D142R/E146R/G157R/C204D/V211W/Q253V/R267A/T327Y, A25F/C75V/T114H/N137E/T141R/D142R/E146R/G157R/C204D/V211W/Q253V/R267A/T327Y, A25F/C75E/T114H/N137V/T141R/G157R/C204D/V211W/Q253V/T327Y, A25F/C75E/T114H/N137V/T141R/D142R/E146R/G157R/C204D/V211W/Q253V/T327Y, A25F/T114H/N137E/T141R/D142R/G157R/C204 D/V211W/Q253V/R267A/T327Y, A25F/T114H/N137V/D142R/G157R/C204D/V211W/Q253V/T327Y, A25F/C75V/T114H/N137V/D142R/G157R/C204 D/V211W/Q253V/T327Y, A25F/C75V/T114H/N137V/D142R/G157R/V211W/Q253V/T327Y, A25F/T114H/N137V/C204D/V211W/T327Y.

46. The variant phytase of claim 35, wherein the mature part of the phytase of SEQ ID NO:2, SEQ ID NO:3, SEQ ID NO:4, SEQ ID NO:5, or SEQ ID NO:6 is used as a parent/backbone for producing a phytase variant.

47. A composition comprising the phytase variant of claim 35, and
(a) at least one fat soluble vitamin;
(b) at least one water soluble vitamin; and/or
(c) at least one trace mineral.

48. An animal feed composition having a crude protein content of 50 to 800 g/kg and comprising the phytase variant of claim 35.

49. A process for reducing phytate levels in animal manure comprising feeding an animal with an effective amount of the feed composition of claim 48.

50. A method for the treatment of a vegetable protein, comprising adding the phytase variant of claim 35 to the vegetable protein.

51. A method for producing a fermentation product comprising fermenting a carbohydrate material in the presence of the phytase variant of claim 35.


SEQUENCE LISTING (6 sequences)

SEQ ID NO: 1 - DNA, 1239 bp, Escherichia coli
  CDS (1)..(1239)
cagagtgagccggagctgaagctggaaagtgtggtgattgtcagtcgtcatggtgtgcgtgctccaaccaaggccacgcaactgatgcaggatgtcaccccagacgcatggccaacctggccggtaaaactgggttggctgacaccgcgcggtggtgagctaatcgcctatctcggacattaccaacgccagcgtctggtagccgacggattgctggcgaaaaagggctgcccgcagtctggtcaggtcgcgattattgctgatgtcgacgagcgtacccgtaaaacaggcgaagccttcgccgccgggctggcacctgactgtgcaataaccgtacatacccagacagatacgtccagtcccgatccgttatttaatcctctaaaaactggcgtttgccaactggataacgcgaacgtgactgacgcgatcctcagcagggcaggagggtcaattgctgactttaccgggcatcggcaaacggcgtttcgcgaactggaacgggtgcttaattttccgcaatcaaacttgtgccttaaacgtgagaaacaggacgaaagctgttcattaacgcaggcattaccatcggaactcaaggtgagcgccgacaatgtctcattaaccggtgcggtaagcctcgcatcaatgctgacggagatatttctcctgcaacaagcacagggaatgccggagccggggtggggaaggatcaccgattcacaccagtggaacaccttgctaagtttgcataacgcgcaattttatttgctacaacgcacgccagaggttgcccgcagccgcgccaccccgttattagatttgatcaagacagcgttgacgccccatccaccgcaaaaacaggcgtatggtgtgacattacccacttcagtgctgtttatcgccggacacgatactaatctggcaaatctcggcggcgcactggagctcaactggacgcttcccggtcagccggataacacgccgccaggtggtgaactggtgtttgaacgctggcgtcggctaagcgataacagccagtggattcaggtttcgctggtcttccagactttacagcagatgcgtgataaaacgccgctgtcattaaatacgccgcccggagaggtgaaactgaccctggcaggatgtgaagagcgaaatgcgcagggcatgtgttcgttggcaggttttacgcaaatcgtgaatgaagcacgcataccggcgtgcagtttgcgtagttaa

SEQ ID NO: 2 - protein, 412 aa, Escherichia coli
QSEPELKLESVVIVSRHGVRAPTKATQLMQDVTPDAWPTWPVKLGWLTPRGGELIAYLGHYQRQRLVADGLLAKKGCPQSGQVAIIADVDERTRKTGEAFAAGLAPDCAITVHTQTDTSSPDPLFNPLKTGVCQLDNANVTDAILSRAGGSIADFTGHRQTAFRELERVLNFPQSNLCLKREKQDESCSLTQALPSELKVSADNVSLTGAVSLASMLTEIFLLQQAQGMPEPGWGRITDSHQWNTLLSLHNAQFYLLQRTPEVARSRATPLLDLIKTALTPHPPQKQAYGVTLPTSVLFIAGHDTNLANLGGALELNWTLPGQPDNTPPGGELVFERWRRLSDNSQWIQVSLVFQTLQQMRDKTPLSLNTPPGEVKLTLAGCEERNAQGMCSLAGFTQIVNEARIPACSLRS

SEQ ID NO: 3 - protein, 412 aa, Escherichia coli
  mat_peptide (1)..(412)
MAQSEPELKLESVVIVSRHGVRAPTKATQLMQDVTPDAWPTWPVKLGWLTPRGGELIAYLGHYQRQRLVADGLLAKKGCPQSGQVAIIADVDERTRKTGEAFAAGLAPDCAITVHTQADTSSPDPLFNPLKTGVCQLDNANVTDAILSRAGGSIADFTGHRQTAFRELERVLNFPQSNLCLKREKQDESCSLTQALPSELKVSADNVSLTGAVSLASMLTEIFLLQQAQGMPEPGWGRITDSHQWNTLLSLHNAQFYLLQRTPEVARSRATPLLDLIKTALTPHPPQKQAYGVTLPTSVLFIAGHDTNLANLGGALELNWTLPGQPDNTPPGGELVFERWRRLSDNSQWIQVSLVFQTLQQMRDKTPLSLNTPPGEVKLTLAGCEERNAQGMCSLAGFTQIVNEARIPACSL

SEQ ID NO: 4 - protein, 412 aa, Escherichia coli
  VARIANT (1)..(412): Variant Nov9X from WO 2009/073399
MAQSEPELKLESVVIVSRHGVRAPTKATQLMQDVTPDAWPTWPVKLGELTPRGGELIAYLGHYWRQRLVADGLLPKCGCPQSGQVAIIADVDERTRKTGEAFAAGLAPDCAITVHTQADTSSPDPLFNPLKTGVCQLDNANVTDAILERAGGSIADFTGHYQTAFRELERVLNFPQSNLCLKREKQDESCSLTQALPSELKVSADCVSLTGAVSLASMLTEIFLLQQAQGMPEPGWGRITDSHQWNTLLSLHNAQFDLLQRTPEVARSRATPLLDLIKTALTPHPPQKQAYGVTLPTSVLFIAGHDTNLANLGGALELNWTLPGQPDNTPPGGELVFERWRRLSDNSQWIQVSLVFQTLQQMRDKTPLSLNTPPGEVKLTLAGCEERNAQGMCSLAGFTQIVNEARIPACSL

SEQ ID NO: 5 - protein, 432 aa, Escherichia coli
  PROPEP (1)..(22)
  mat_peptide (23)..(432)
MKAILIPFLSLLIPLTPQSAFAQSEPELKLESVVIVSRHGVRAPTKATQLMQDVTPDAWPTWPVKLGELTPRGGELIAYLGHYWRQRLVADGLLPKCGCPQSGQVAIIADVDERTRKTGEAFAAGLAPDCAITVHTQADTSSPDPLFNPLKTGVCQLDNANVTDAILERAGGSIADFTGHYQTAFRELERVLNFPQSNLCLKREKQDESCSLTQALPSELKVSADCVSLTGAVSLASMLTEIFLLQQAQGMPEPGWGRITDSHQWNTLLSLHNAQFDLLQRTPEVARSRATPLLDLIKTALTPHPPQKQAYGVTLPTSVLFIAGHDTNLANLGGALELNWTLPGQPDNTPPGGELVFERWRRLSDNSQWIQVSLVFQTLQQMRDKTPLSLNTPPGEVKLTLAGCEERNAQGMCSLAGFTQIVNEARIPACSL

SEQ ID NO: 6 - protein, 432 aa, Escherichia coli
  PROPEP (1)..(22)
  mat_peptide (23)..(432)
MKAILIPFLSLLIPLTPQSAFAQSEPELKLESVVIVSRHGVRAPTKATQLMQDVTPDAWPTWPVKLGWLTPRGGELIAYLGHYQRQRLVADGLLAKKGCPQSGQVAIIADVDERTRKTGEAFAAGLAPDCAITVHTQADTSSPDPLFNPLKTGVCQLDNANVTDAILSRAGGSIADFTGHRQTAFRELERVLNFPQSNLCLKREKQDESCSLTQALPSELKVSADNVSLTGAVSLASMLTEIFLLQQAQGMPEPGWGRITDSHQWNTLLSLHNAQFYLLQRTPEVARSRATPLLDLIKTALTPHPPQKQAYGVTLPTSVLFIAGHDTNLANLGGALELNWTLPGQPDNTPPGGELVFERWRRLSDNSQWIQVSLVFQTLQQMRDKTPLSLNTPPGEVKLTLAGCEERNAQGMCSLAGFTQIVNEARIPACSL